(19) United States
(12) Patent Application Publication
(10) Pub. No.: US 2008/0311657 A1
(43) Pub. Date: Dec. 18, 2008

(54) Killing Human Lymphoma and Leukemia Cancer Cells and Tcr-Activated Normal Human Cells By Dopamine D1r Agonists

(76) Inventor: Mia Levite, Savyon (IL)

Correspondence Address: BROWDY AND NEIMARK, P.L.L.C.;624 NINTH STREET, NW, SUITE 300, WASHINGTON, DC, 20001-5303, US

(21) Appl. No.: 11/997,848

(22) PCT Filed: Aug. 3, 2006
(86) PCT No.: PCT/US2006/030360

§ 371 Date: Feb. 4, 2008

Related U.S. Application Data
(60) Provisional application No. 60/704,728, filed on Aug. 3, 2005.

(51) Int. Cl. C12N 5/08; C12N 5/02

(52) U.S. Cl. 435/372.3; 435/366; 435/375; 435/372

(57) ABSTRACT

The dopamine D1/D5 receptor is highly over-expressed in various types of human and animal leukemia, lymphoma and activated T-cells. The dopamine D1 receptor is also expressed in dramatically elevated or even moderate levels in other types of cancer cells. Selective dopamine D1 receptor agonists, such as fenoldopam mesylate, rapidly, potently and selectively kill such human and animal T-cells expressing the dopamine D1 receptor. Thus, selective dopamine D1/5 receptor agonists may be used to treat lymphoma, leukemia and other cancers of the immune system, and T-cell mediated autoimmune diseases and other diseases caused by over-activated inflammatory T-cells (such as chronic inflammation), or graft versus host diseases (GVHD) or graft rejection, or by any other cell types expressing the dopamine D1 receptor, by killing the disease-causing cells. The selective dopamine D1/5 receptor agonists can be used for these purposes either in vivo or in vitro, such as to purge a given cell population from undesired leukemia, lymphoma or activated T-cells prior to further use.

### BACKGROUND OF INVENTION

### Lymphoma and Leukemia

Humans suffer from various types of lymphoma and leukemia, which are very aggressive tumors. In the majority of the cases, the currently existing treatment modalities (chemotherapy, radiotherapy, surgery, certain additional anti-cancer drugs and bone marrow transplantation) are far from satisfactory, and only a relatively small proportion of lymphoma and leukemia patients can survive for many years. Thus, there is an urgent need to find novel drugs that can kill selectively leukemia and lymphoma cancer cells, while affecting to a much lesser extent, if at all, normal (non malignant) cells.

### Dopamine and its Receptors

Dopamine, one of the most important neurotransmitters in the nervous system, has five receptors, DR1-DR5, subdivided into the D1R-family, which consists of the D1R and D5R, and the D2R-family, which consists of the D2R, D3R and D4R. The D1 class of dopamine receptors, (again, to which the D1R and D5R belong), are Gs protein coupled, whereas the D2 class of dopamine receptors, (again, to which the D2R, D3R and D4R belong), are Gi coupled.

Several independent studies show that normal human T cells and peripheral lymphocytes express dopaminergic receptors of the D2, D3, D4 and D5 subtypes, but not the dopamine D1 receptor subtype.

### Fenoldopam Mesylate

Fenoldopam mesylate is a highly selective Dopamine D1 receptor agonist, extensively studied and used in the clinic for its vasodilatory actions, mainly in the treatment of severe hypertension, congestive heart failure, and acute and chronic renal failure.

Fenoldopam mesylate does not cross the BBB, and thus has only peripheral actions. Chemically, fenoldopam is 6 chloro-2,3,4,5-tetrahydro-1-(4-hydroxyphenyl)-[1H]-3-benzazepine-7,8-diol methanesulfonate. It has been described in U.S. Pat. Nos. 4,197,297, 4,600,714 and 6,238,693 and is now a generic drug.

Fenoldopam is a racemic mixture with the R-isomer responsible for the biological activity. The R-isomer has approximately 250-fold higher affinity for D1-like receptors than does the S-isomer. Fenoldopam binds but with moderate affinity to α2-adrenoceptors. It has no significant affinity for D2-like receptors, α1 and β adrenoceptors, 5HT1 and 5HT2 receptors, or muscarinic receptors. There has been so far no evidence that fenoldopam or any other D1 receptor agonist has the ability to kill cancer cells. It has now been found that various types of human and animal leukemia and lymphoma, as well as activated T-cells, express highly elevated levels of dopamine D1 receptor as compared to normal resting T-cells that do not express the D1 receptor. It has also been found that fenoldopam, a selective dopamine D1 receptor agonist and other selective dopamine D1 receptor agonists rapidly, potently and selectively kill lymphoma, leukemia and activated T-cells. Based on these findings, the present invention is directed to the use of fenoldopam mesylate and other dopamine D1 receptor agonists to selectively kill leukemia, lymphoma, activated T-cells, autoimmune T-cells and over-activated inflammatory T-cells. It is expected that fenoldopam also has the ability to kill other cancer cells that express the dopamine D1 receptor.

### T-Cell Mediated Autoimmune Diseases

Humans suffer from several types of autoimmune diseases, some of which are mediated (to a greater or lesser extent) by autoimmune T-cells. Among the human T-cell mediated autoimmune diseases are the following: insulin-dependent (type 1) diabetes mellitus, multiple sclerosis, myasthenia gravis, autoimmune myocarditis, and probably also, at least in part (according to novel observations made in recent years) alopecia and psoriasis. The beneficial outcome of the existing treatments of all these diseases is very limited and far from satisfactory. Thus, there is an urgent need to find novel drugs that can kill or silence selectively activated autoimmune T-cells, while sparing resting non-activated T-cells.

### SUMMARY OF THE INVENTION

The aspect of the present invention relating to the killing of lymphomas and leukemias is based on the following findings:

1) Some types of human and mouse lymphoma (among them several types of T-cell lymphoma and leukemia (among them T-cell leukemia) have dramatic elevation in the levels of dopamine D1 receptors expressed on their cell surface, in contrast to normal human resting peripheral T-cells, which do not express the D1 dopamine receptors. Other types of non T-leukemia and lymphoma (among them B-cell Burkett's lymphoma) also express various levels of the dopamine D1 receptor.

2) Exposing in vitro five different types of human lymphoma and leukemia (specified above) to concentrations of 1 mM-0.01 mM of fenoldopam mesylate or to similar concentrations of other dopamine D1/5 receptor agonists leads to the death of all or the vast majority of these cancer cells.

3) Exposing different types of human lymphoma and leukemia for relatively short time periods (e.g., 10-30 minutes) in vitro to fenoldopam mesylate or to other highly specific dopamine D1/5 receptor agonists (specified below) is enough to cause the death of lymphoma or leukemia cells. The selective dopamine D1/5 receptor agonists tested and found effective in killing lymphoma and leukemia are: (1R-cis)-1-(aminomethyl)-3,4-dihydro-3-tricyclo[3.3.1.13,7]dec-1-yl-[1H]-2-benzopyran-5,6-diol hydrochloride (TOCRIS Cookson Product name: A 77636 hydrochloride; Catalogue number: 1701; referred to as “potent, selective D1-like agonist; orally active”), (±)-1-phenyl-2,3,4,5-tetrahydro-(1H)-3-benzazepine-7,8-diol hydrobromide (TOCRIS COOKSON Product name: SKF 38393 hydrobromide; Catalogue number: 0922; referred to as “D1-like dopamine receptor selective partial agonist”), and cis-(±)-1-(aminomethyl)-3,4-dihydro-3-phenyl-1H-2-benzopyran-5,6-diol hydrochloride (TOCRIS COOKSON Product name: 1534; Catalogue number: A 68930 hydrochloride; referred to as “potent and selective D1-like dopamine receptor agonist”).

4) The killing of lymphoma and leukemia by fenoldopam mesylate and all the other selective dopamine D1/5 receptor agonists was always dose dependent. Nevertheless, as expected, some D1R agonists were much more effective than others, and could kill the cancer cells in lower concentrations than the others. Fenoldopam melylate and A 77636 hydrochloride were the most effective cancer killers and are thus preferred embodiments for use the present invention.

5) Most of the lymphoma and leukemia cells tested expressed on their cell surface markedly elevated levels not only of the D1/5 receptor, but also of the dopamine D3 and dopamine D2 receptors, compared to much lower expression of the respective receptors on normal (not cancer) human T-cells. Yet, dopamine D2 and D3 receptor agonists, exhibited much lower anti-cancer killing activity, if at all, compared to the effect exerted by the dopamine D1/5 receptor agonists.

While the dopamine D1R agonists consistently caused substantial death, primarily by necrosis, of the leukemia and lymphoma cells tested, dopamine itself, that in principle can trigger all of its five receptor subtypes) in some cases also killed the human leukemia and lymphoma, but in some other cases failed to do so. Of all the highly selective D1R agonists tested herein, fenoldopam mesylate and A 77636 hydrochloride were the most effective cancer killers.

The aspect of the present invention related to the treatment of T-cell mediated autoimmune diseases is based on the findings that:

1. T-cell receptor (TCR)-activated normal human peripheral T-cells express dramatically elevated levels of dopamine D1 receptors on their cell surface (as opposed to resting normal human peripheral T-cells that do not express this receptor, or do so to minimal not significant levels).

2. Exposing TCR-activated human normal peripheral T-cells in vitro to several highly selective dopamine D1/5 receptor agonists, such as fenoldopam mesylate, kills a substantial proportion of these activated T-cells, but significantly less of the resting (not activated) human normal peripheral T-cells. The killing of TCR-activated T-cells by all the selective dopamine D1/5 receptor agonists was dose dependent. Nevertheless, as expected, some D1R agonists were much more effective than others, and could kill the cancer cells in lower concentrations than the others. Of all the highly selective D1R agonists tested herein, fenoldopam mesylate and A 77636 hydrochloride were the most effective killers of TCR-activated T-cells and are thus the preferred embodiments for use in this method.

### DESCRIPTION OF THE DRAWINGS

The present invention will be better understood with reference to the attached drawings, in which:

FIG. 1A-F show flow cytometry FACSort results establishing that dopamine D1 receptor is expressed in the vast majority of T-leukemia and T-lymphoma cells, but hardly in normal human T-cells. In FIGS. 1A-C, freshly isolated normal human T-cells, as well as human T-leukemia cell line (Jurkat) and mouse T-lymphoma cell line (EL-4) were subjected to double immunofluorescence staining using the rabbit anti-DR1 IgG, followed by FITC-conjugated anti-rabbit IgG (second Ab) and PE-conjugated anti-human TCRαβ mAb (third Ab) (the latter to confirm the T-cell origin of all the tested cells). In FIGS. 1D-F, isotype control non specific staining of all three types of T-cells, using normal rabbit serum and similar second and third Abs. The actual percentage of TCR⁺D1R⁺ double positive cells within each of the T-cell types, was deduced by subtracting the non specific staining (framed window of each lower figure) from the specific staining (framed window of each, upper figure): Normal human T-cells: % TCR⁺D1R⁺ cells=13.9−8.21=5.69%; human T-cell leukemia: % TCR⁺D1R⁺ cells=74.8−13.7=61.1%; mouse T-cell lymphoma: % TCR⁺D1R⁺ cells=71−13.2=57.8%. Representative experiment out of 4 performed.

FIG. 2 is a graph showing that fenoldopam mesylate (FDM), a highly selective dopamine D1R agonist, kills human T-cell leukemia in a dose dependent manner. Human T-cell leukemia (Jurkat) were seeded in 96 well plates (0.5 ml/well of 0.2 million cells/ml), and FDM, at starting concentrations of 10⁻² M-10⁻¹² M, was added and diluted 1:00 into the corresponding wells, so that the final FDM concentration range tested was 10⁻⁴ M-10⁻¹² M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of lactate dehydrogenase (LDH), a stable cytosolic enzyme that is released upon cell death/lysis, was measured with a commercial kit, according to the manufacturer's instruction, and as described in the Materials and Methods (Example 1).

FIG. 3 is a graph showing that fenoldopam mesylate (FDM), kills human cutaneous Sezary T-cell lymphoma in a dose dependent manner. Human Sezary T-cell lymphoma cells (HUT-78) were seeded in 96 well plates (0.5 ml/well of 0.2 million cells/ml), and FDM, at starting concentrations of 10⁻²M-10⁻¹⁰M, was added and diluted 1:00 into the corresponding wells, so that the final FDM concentration range tested was 10⁻⁴M-10⁻¹²M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIG. 4 is a graph showing that FDM kills human chronic myelogenous leukemia (CML) in a dose dependent manner. Human CML (K-562) cells were seeded in 96 well plates (0.5 ml/well of 0.2 million cells/ml), and FDM, at starting concentrations of 10⁻²M-10⁻¹⁰M, was added and diluted 1:00 into the corresponding wells, so that the final FDM concentration range tested was 10⁻⁴M-10⁻¹²M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIG. 5 is a graph showing that FDM kills human Burkitt's B-lymphoma in a dose dependent manner. Human Burkitt's B-lymphoma cells (Daudi) were seeded in 96 well plates (0.5 ml/well of 0.2 million cells/ml), and FDM, at starting concentrations of 10⁻²M-10⁻¹⁰M, was added and diluted 1:00 into the corresponding wells, so that the final FDM concentration range tested was 10⁻⁴M-10⁻¹²M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIGS. 6A and B are graphs showing that dopamine D1 receptor is expressed in the vast majority of human TCR-activated (FIG. 6B), but not in resting, normal (FIG. 6A) peripheral T-cells. Normal human T-cells, purified from a “fresh” blood sample of an arbitrary individual, were either not treated any further and left as such for 72 hr incubation in a humidified incubator, or underwent “classical” T-cell receptor (TCR) activation in vitro (using anti-CD3 and anti-CD 28 monoclonal antibodies, as described in the material and methods) (FIG. 6B). Then, the “resting” and the TCR-activated T-cells were subjected to single immunofluorescence staining using the rabbit anti-DR1 IgG, followed by FITC-conjugated anti-rabbit IgG (second Ab) (FIG. 6A). In parallel, the cells were subjected to non specific control staining, using normal rabbit serum, instead of the anti-D1R antibody (also shown as alternative lines in FIGS. 6A and 6B).

FIGS. 7A and B are graphs showing that dopamine D1 receptor is expressed in the vast majority of human TCR-activated (FIG. 7B) but not in resting, normal (FIG. 7A) peripheral T-cells. Normal human T-cells, purified from a “fresh” blood sample of another arbitrary individual, were treated and tested exactly as described in FIG. 6.

FIG. 8 is a graph showing that FDM kills human TCR-activated T-cells, in a dose dependent manner. Normal human T-cells, purified from a “fresh” blood sample for a given arbitrary individual, were either left as such or underwent “classical” T-cell receptor (TCR) activation in vitro (using anti-CD3 and anti-CD28 monoclonal antibodies, as described in the material and methods). Then, both the TCR-activated T-cells and the resting untreated cells (results shown in FIG. 9) were seeded in 96 well plates (0.5 ml/well of 0.2 million cells/ml), and FDM, at starting concentrations of 10⁻²M-10⁻⁸M, was added and diluted 1:00 into the corresponding wells, so that the final FDM concentration range tested was 10⁻⁴M-10⁻¹⁰M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIG. 9 is a graph showing that FDM has a significantly milder killing effect on resting normal human T-cells. Normal human T-cells, purified from a “fresh” blood sample for a given arbitrary individual, were either left as such (and thus considered “resting”) or underwent “classical” T-cell receptor (TCR) activation in vitro (using anti-CD3 and anti-CD28 monoclonal antibodies, as described in the material and methods). Then, both the TCR-activated T-cells (results shown in FIG. 8) and the resting untreated cells were seeded in 96 well plates (0.5 ml/well of 0.2 million cells/ml), and FDM, at starting concentrations of 10⁻²M-10⁻⁸M, was added and diluted 1:00 into the corresponding wells, so that the final FDM concentration range tested was 10⁻⁴M-10⁻¹⁰M. FDM (at each of the above mentioned concentrations) was added to the corresponding microtiter well four times during 1 hour total, at time 0, 15 min, 30 min and 45 min. In between these additions of FDM, the microtiter plates were placed in a humidified incubator (37° C., with 5% CO₂). Fifteen minutes after the last addition of FDM, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIG. 10 is a graph showing that the highly selective dopamine D1R agonist, A 77636 hydrochloride, induces marked cell death of human T-cell leukemia, in a dose dependent manner. Human T-cell leukemia (Jurkat) cells were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested). Of note, A 77636 hydrochloride is an orally-active D1R agonist, according to the manufacturer (Tocris).

FIG. 11 is a graph showing that the highly selective dopamine D1R agonist, A 68930 hydrochloride, induces marked cell death of human T-cell leukemia, in a dose dependent manner. Human T-cell leukemia (Jurkat) cells were seeded in 96 well plates (0.5 ml per well of 0.5 million cells/ml) and A 68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 12 is a graph showing that the highly selective dopamine D1R agonist, SKF 38393 hydrobromide, induces marked cell death of human T-cell leukemia, in a dose dependent manner. Human T-cell leukemia (Jurkat) cells were seeded in 96 well plates (0.5 ml per well of 0.5 million cells/ml) and SKF-38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 13 is a graph showing that A 77636 hydrochloride induces marked cell death of human-cutaneous Sezary T-lymphoma, in a dose dependent manner. Human cutaneous Sezary T-lymphoma cells (HUT-78) were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 14 is a graph showing that A 68930 hydrochloride induces marked cell death of human cutaneous Sezary T-lymphoma, in a dose dependent manner. Human cutaneous Sezary T-lymphoma cells (HUT-78) were seeded in 96 well plates (0.5 ml per well of 0.5 million cells/ml) and A 68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 15 is a graph showing that SKF 38393 hydrobromide induces marked cell death of human cutaneous Sezary T-lymphoma, in a dose dependent manner. Human cutaneous Sezary T-lymphoma cells (HUT-78) were seeded in 96 well plates (0.5 ml per well of 0.5 million cells/ml) and SKF 38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 16 is a graph showing that A 77636 hydrochloride induces marked cell death of human Burkitt's B-lymphoma, in a dose dependent manner. Human Burkitt's B-lymphoma cells (Daudi) were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 17 is a graph showing that A 68930 hydrochloride induces marked cell death of human Burkitt's B-lymphoma, in a dose dependent manner. Human Burkitt's B-lymphoma cells (Daudi) were seeded in 96 well plates (0.5 ml per well of 0.5 million cells/ml) and A 68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 18 is a graph showing that SKF 38393 hydrobromide induces marked cell death of human Burkitt's B-lymphoma, in a dose dependent manner. Human Burkitt's B-cell lymphoma (Daudi) cells were seeded in 96 well plates (0.5 ml per well of 0.5 million cells/ml) and SKF 38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 19 is a graph showing that A 77636 hydrochloride induces marked cell death of human Burkitt's B-lymphoma, in a dose dependent manner. Human Burkitt's B-cell lymphoma (Raji) cells were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M 10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 20 is a graph showing that A 68930 hydrochloride induces marked cell death of human Burkitt's B-lymphoma, in a dose dependent manner. Human Burkitt's B-cell lymphoma (Raji) cells were seeded in 96 well plates (0.5 ml per well of 0.5 million cells/ml) and A 68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 21 is a graph showing that SKF 38393 hydrobromide induces marked cell death of human Burkitt's B-lymphoma, in a dose dependent manner. Human Burkitt's B-cell lymphoma (Raji) cells were seeded in 96 well plates (0.5 ml per well of 0.5 million cells/ml) and SKF 38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 22 is a graph showing that A 77636 hydrochloride induces marked cell death of chronic myelogenous leukemia, in a dose dependent manner. Human chronic myelogenous leukemia cells (CML) (K-562) were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 23 is a graph showing that A 68930 hydrochloride induces marked cell death of chronic myelogenous leukemia, in a dose dependent manner. Human chronic myelogenous leukemia cells (CML) (K-562) were seeded in 96 well plates (0.5 ml per well of 0.5 million cells/ml) and A 68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 24 is a graph showing that SKF 38393 hydrobromide induces marked cell death of chronic myelogenous leukemia, in a dose dependent manner. Human chronic myelogenous leukemia cells (CML) (K-562) were seeded in 96 well plates (0.5 ml per well of 0.5 million cells/ml) and SKF 38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 25 is a graph showing that A 77636 hydrochloride has a significantly milder killing effect on resting normal human T-cells. Normal human T-cells, purified from a “fresh” blood sample of another arbitrary individual, were seeded in 96 well plates (0.5 ml per well of 0.5 million cells/ml) and A 77636 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 26 shows A 68930 hydrochloride has a significantly milder killing effect on resting normal human T-cells. Normal human T-cells, purified from a “fresh” blood sample of another arbitrary individual, were seeded in 96 well plates (0.5 ml per well of 0.5 million cells/ml) and A-68930 hydrochloride was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 27 shows SKF 38393 hydrobromide has a significantly milder killing effect on resting normal human T-cells. Normal human T-cells, purified from a “fresh” blood sample of another arbitrary individual, were seeded in 96 well plates (0.5 ml per well of 0.5 million cells/ml) and SKF 38393 hydrobromide was added and diluted 1:00 into the wells at starting concentrations of 10⁻¹M-10⁻⁴M, so that the final concentration range tested was 10⁻³M-10⁻⁶M. Afterwards, the microtiter plates were placed in an incubator (37° C., humidified incubator, 5% CO₂) for 3 days. Then, the number of living cells was evaluated by flow cytometry (the cells were counted by FACsort for a fixed time length of 1 min, in which 100 microliter of each sample was tested).

FIG. 28 shows A 77636 hydrochloride causes a very rapid death of human Burkitt's B-lymphoma. Human Burkitt's B-lymphoma cells (Raji) were seeded in 96 well plates (0.5 ml per well of 0.5 million cells/ml) and A 77636 hydrochloride was added and diluted 1:00 into the wells, at a fixed starting concentration of 10⁻²M, so that the final concentration tested was 10⁻⁴M. The cells were then transferred to an incubator (37° C., humidified incubator, 5% CO₂) for 1 min, 10 min, 30 min, 60 min or 120 min incubation. Then, 50 microliter supernatant was removed carefully from the upper part of each well, and the extent of release into this supernatant of LDH, a stable cytosolic enzyme that is released upon cell death/lysis, was measured with a commercial kit, according to the manufacturer's instruction, and as described in the Materials and Methods (Example 1).

FIG. 29 is a graph showing that A 77636 hydrochloride causes a very rapid death of human chronic myelogenous leukemia. Human chronic myelogenous leukemia cells (CML) (K-562) were seeded in 96 well plates (0.5 ml per well of 0.5 million cells/ml) and A 77636 hydrochloride was added and diluted 1:00 into the wells, at a fixed starting concentration of 10⁻²M, so that the final concentration tested was 10⁻⁴M. The experiment was designed to test the effect of exposing the cells to the D1R agonist for 1 min, 15 min, 1 hr or 72 hr. Thus, 1 min, or 15 min or 1 hr after the addition of the D1R agonist, the corresponding cells were transferred into tubes, centrifuged (1000 rpm for 10 min), and the supernatant was removed. The cells were then resuspended in fresh media (i.e. which did not contain the D1R agonist), seeded in new clean microtiter wells, and returned to the incubator for additional 3 days. The 72 hr sample did not undergo such centrifugation after the addition of the D1R agonist. Thus, its medium was not replaced, and these cells and remained as such in the incubator for 72 hr. At the end of the 72 hr incubation, the number of living cells was evaluated by flow cytometry (the cells were counted by FACsort for a fixed time length of 1 min, in which 100 microliter of each sample was tested).

FIG. 30 shows A 77636 hydrochloride causes a very rapid death of human T-cell leukemia. Human T-leukemia (Jurkat) cells were seeded in 96 well plates (0.5 ml per well of 0.5 million cells/ml) and A 77636 hydrochloride was added and diluted 1:00 into the wells, at a fixed starting concentration of 10⁻²M, so that the final concentration tested was 10⁻⁴M. The experiment was designed to test the effect of exposing the cells to the D1R agonist for 1 min, 15 min, 1 hr or 72 hr. Thus, 1 min, or 15 min or 1 hr after the addition of the D1R agonist, the corresponding cells were transferred into tubes, centrifuged (1000 rpm for 10 min); and the supernatant was removed. The cells were then resuspended in fresh media (i.e., which did not contain the D1R agonist), seeded in new clean microtiter wells, and returned to the incubator for additional 3 days. The 72 hr sample did not undergo such centrifugation after the addition of the D1R agonist. Thus, its medium was not replaced, and these cells and remained as such in the incubator for 72 hr. At the end of the 72 hr incubation, the number of living cells was evaluated by flow cytometry (the cells were counted by FACsort for a fixed time length of 1 min, in which 100 microliter of each sample was tested).

FIGS. 31A and B are graphs showing that A 77636 hydrochloride kills much more TCR-activated (FIG. 31B) than resting normal (FIG. 31A) human T-cells. Normal human T-cells, purified from a “fresh” blood sample for a given arbitrary individual, were either left as such or underwent “classical” T-cell receptor (TCR) activation in vitro (using anti-CD3 and anti-CD28 monoclonal antibodies, as described in the material and methods). Then, both the TCR-activated T-cells (FIG. 31B) and the resting untreated cells (FIG. 31A) were seeded in 96 well plates (0.5 ml/well of 0.2 million cells/ml), and a highly selective dopamine D1R agonists: A 77636 hydrochloride, was added at the final concentration of 10⁻⁵M. The cells were then transferred to the incubator for 72 hr incubation. At the end of the 72 hr incubation, the number of living cells in each well was evaluated by flow cytometry (the cells were counted by FACsort for a fixed time length of 1 min, in which 100 microliter of each sample was tested).

### DETAILED DESCRIPTION OF PREFERRED EMBODIMENTS

While five different selective D1R agonists are specifically disclosed herein and used in the experiments, the present invention is not to be considered limited thereto. It is within the skill of the art to determine other such agonists, such as by varying the structures of the molecules which are known to be such agonists and screening for agonistic activity or by other means known in the art. Additionally, monoclonal antibodies often have agonistic activity.

Accordingly, antibodies can be raised using D1R, or epitopes thereof, as antigen and screened for D1R agonistic activity. Any such positive antibody can then be used directly in accordance with the present invention or genetically engineered in conventional ways to produce humanized antibodies, single chain antibodies, or antibody fragments or derivatives that retain the D1R agonizing activity of the parent antibody. The term “antibody” as used herein is intended to include polyclonal or monoclonal antibodies or any of the aforementioned genetically engineered antibodies.

The dopamine D1 agonist may activate the dopamine D1 receptor directly or indirectly. The G-protein linked protein of the receptor or any of its downstream effector proteins may also be directly or indirectly activated by means of the agonists of the present invention. Once the effect of the present invention is understood, it is within the skill of one of ordinary skill in the art to screen for and obtain other agonists having the desired activity and selectivity.

The term “selective” as used in the present specification and claims means having substantially selective agonist activity against the D1R and D5R with comparatively little or no activity against the D2R, D3R and D4R. While the agonists of the present invention are preferably totally selective for the dopamine D1 receptor, it is permissible that they also have some agonist activity against the D5 receptor, which is also a member of the D1 family of dopamine receptors. Preferred agonists have strong activity with respect to the D1R and as little activity as possible against the D5R, with comparatively little or no activity against the D2R, D3R and D4R.

Any cell that expresses the dopamine D1 receptor, particularly those that over-express such receptor, may be killed by means of the present invention. As indicated herein, certain leukemia and lymphoma cells (often 70-80% positive for D1R) and TCR-activated cells over-express the D1R as compared to the corresponding normal or resting cells. Yet, some other cancers have much lower D1R expression (sometimes even only 10% positive), but are also killed very effectively by the D1R agonists in accordance with the present invention. Thus, even low or moderate levels of D1R may make the cells susceptible to death induced by D1R selective agonists. Accordingly, the present invention is intended also to cover the killing of other malignant cells that express the D1R at even low or moderate levels.

TCR-activated T-cells over-express D1R as compared to normal “resting” T-cells. Thus, such activated cells may be eliminated in diseases or conditions in which said activated T-cells contribute to the disease or condition to be treated, i.e., the disease or condition is caused or exacerbated by activated T-cells, such as inflammatory T-cells. Examples of such diseases or conditions are T-cell mediated autoimmune diseases, such as insulin-dependent (type 1) diabetes mellitus, multiple sclerosis, myasthenia gravis, autoimmune myocarditis, alopecia and psoriasis. Other such diseases include intractable inflammation and other diseases mediated by inflammatory T-cells.

Another disease or condition treatable in accordance with the present invention is graft versus host disease (GVHD). GVHD may be prevented or treated by killing the activated host activated allogeneic T-cells coming from the human and/or animal donor. Such activated T-cells can otherwise cause GVHD subsequent to a transplantation of fully or partially mismatched organ or bone marrow cells. Similarly, graft rejection can be treated or prevented by means of the present invention. Activated host T-cells may cause a host reaction against the donor tissue thereby resulting in graft rejection subsequent to transplantation of fully or partially mismatched organ or bone marrow cells.

The agonists of the present invention may be used to cause the death of cells expressing the D1R receptor either in vivo or in vitro. When treating a disease in a human or other animal subject, the agonist of the present invention may be administered systemically in any convenient manner known in the art or locally to the situs of the cells to be treated. Thus, the agonists may be administered by intravenous, subcutaneous, intraperitoneal, intratumoral, intrathecal, or intracranial injections. The agonists may be administered by transdermal ointments or an implantable drug-delivery pump. The agonists may also be administered orally.

The agonists of the present invention may also be used ex vivo. For example, they can be used in such a manner to purge and/or kill leukemia and/or lymphoma cells, such as for killing the cancer cells within a preparation of autologous stem cells to be used later for autologous bone marrow transplantation. Indeed, dopamine D1 receptor agonists can be used to purge or “clean” a given cell population, such as bone marrow cells, from undesired leukemia, lymphoma or activated T-cells, before further use of the “cleaned” cell population for bone marrow transplantation, T-cell transplantation, or any other use. Such “cleaned” cell population may also be used, for example for further in vitro culturing such as for immunotherapy of cancer, collecting T-cell cytokines or growth factors or any other T-cell secrete protein, etc.

### Example 1

### Materials and Methods

Dopamine D1 Receptor Agonists Tested for their Anti-Cancer Effects

Five different highly selective dopamine D1/5 receptor agonists were tested for their anti-lymphoma and anti-leukemia killing activity:

1) TOCRIS Cookson Product name: A 77636 hydrochloride; Catalogue number: 1701; Chemical name: (1R-cis)-1-(aminomethyl)-3,4-dihydro-3-tricyclo[3.3.1.13,7]dec-1-yl-[1H]-2-benzopyran-5,6-diol hydrochloride, referred to as “Potent, selective D1-like agonist; Orally active.”

2) TOCRIS COOKSON Product name: SKF 38393 hydrobromide; Catalogue number: 0922; Chemical name: (±)-1-phenyl-2,3,4,5-tetrahydro-(1H)-3-benzazepine-7,8-diol hydrobromide referred to as “D1-like dopamine receptor selective partial agonist.”

3) TOCRIS COOKSON Product name: 1534; Catalogue number: A 68930 hydrochloride; Chemical name: cis-(±)-1-(aminomethyl)-3,4-dihydro-3-phenyl-1H-2-benzopyran-5,6-diol hydrochloride, referred to as “Potent and selective D1-like dopamine receptor agonist.”

4) Fenoldopam Mesylate (FD): Bedford Laboratories/USA product named “Fenoldopam Mesylate injection USP” (fenoldopam is 6-chloro-2,3,4,5-tetrahydro-1-(4-hydroxyphenyl)-[1H]-3-benzazepine-7,8-diol methanesulfonate).

5) Fenoldopam Hydrobromide: SIGMA product number F6800, CAS#: 67227-56-9; Synonyms: SKF 82526.

Dopamine and Other Dopamine-Receptor Analogues were Used as Controls

i. Dopamine and dopamine D3R selective antagonist: U-99194A maleate (Sigma Chemicals). Dopamine D1R selective agonist: SKF 38393. Dopamine-D2R selective agonist: Quinpirole. Dopamine D3R selective agonist: 7-Hydroxy-DPAT;

ii. Dopamine D4R selective agonist: PD 168077. Dopamine D2R selective antagonist: L-741,626. Dopamine D4R selective antagonist: L-741,741 (Tocris Cookson).

### Human Cancer Cell Lines

Human B-lymphoma (Burkitt's lymphoma) lines: Raji and Daudi; human T-cell leukemia line: Jurkat; human T-lymphoma (cutaneous “Sezary” T-lymphoma) line: HuT-78; and human Chronic-Myeloid Leukemia (CML): K-562 were obtained from American Type Cell Culture (ATCC), and maintained (37° C., humidified incubator, 5% CO₂) either in tissue culture medium (either IMDM or RPMI-1640), supplemented with 10% FCS, 1% glutamine and 1% antibiotics.

### Normal Peripheral Human T-Cells

Density gradient centrifugation was used to separate the lymphocytes from the erythrocytes, dead cells, polymorphonuclear leukocytes and granulocytes. A “fresh” 50 ml sample of leukocytes, without plasma and without prior freezing, supplied by the blood bank, was diluted 1:1 in PBS and added to Uni-SEPmaxi+ tubes (Novamed, Jerusalem, Israel) containing at their bottom a solution of 5.6% polysucrose and 9.6% sodium metrizoate. The tubes were centrifuged (1200 rpm, 30 minutes), and the resulting layer of lymphocytes (migrating to the interface between the plasma and polysucrose/sodium metrizoate) was removed by a 2 ml pipette. The lymphocytes were washed twice with PBS (1000 rpm, 10 minutes) and resuspended in 8 ml PBS containing 5% FCS. Nylon wool columns were then used to separate the T-cells from the other lymphocytes (i.e., B-cells and NK-cells). The cell suspension (2 ml per column) was loaded (by syringe injection) on nylon wool columns (Novamed) that have been pre-incubated for 30 minutes at 37° C. with PBS/5% FCS. After this cell loading, the columns were further incubated, lying flat, for 1 hour at room temperature. Following incubation, PBS (12 ml per column) was added to the columns for eluting the non-adherent T-cells. The eluted cells were collected in a clean tube and centrifuged (800 rpm, 15 minutes). The resulting cell population consisted of >90% T-cells, as evaluated by TCR staining and flow cytometry, using FACSort. The cells were maintained (37° C., humidified incubator, 5% CO₂) in RPMI-1640 supplemented with 10% FCS, 1% glutamine and 1% antibiotics.

### T Cell Receptor (TCR) Activation of Normal Peripheral Human T-Cells

Non-tissue culture treated 24-well plates (Falcon, Franklin Lakes, N.J.) were coated overnight at 4° C. with anti-CD3 and anti-CD28 monoclonal antibodies (mabs) (BD Pharmingen, San Jose, Calif.); (10 g/ml in PBS). The wells were then washed with PBS, blocked for 1 hour at 37° C. (PBS/1% BSA), and washed again. The freshly purified normal human T-cells were resuspended in their respective fresh media and seeded in the anti-CD3/CD28 coated wells (1×10⁶ per well), and the plates were incubated for 72 hours (37° C., humidified incubator, 5% CO₂). Then, the cells and their media were collected from each well, transferred into 50 ml tubes, centrifuged (1200 rpm, 10 minutes) and both the TCR-activated cells and their culture media were collected and transferred into clean separate tubes.

Exposure of Cancer Cells, as Well as Normal “Resting” and Normal TCR-Activated T-Cells to D1R Agonists (Among them FD)

Human cancer cells, and in parallel “resting” and T-cell receptor (TCR)-activated normal human T-cells, were seeded in 96 tissue culture wells (0.2.-0.5 million cells/well), and added with D1R agonists at serial dilutions, usually at the range of 0.1 nM-0.1 mM (unless indicated otherwise) for various time periods ranging from 1 minute to 72 hours. Cell viability was tested afterwards. In most experiments with FD, this drug was added again at serial dilutions of 0.01 nM-0.1 mM, four times (FD ×4) during 1 hour total, at time 0, 15 minutes, 30 minutes and 60 minutes.

### Testing the Effect of FD on Cell Viability by Following LDH Release

Measurement of cell death by measuring the release of LDH was performed using The CytoTox 96® Non-Radioactive Cytotoxicity Assay (Promega) according to the manufacturer's instructions.

In detail: The CytoTox 96® Non-Radioactive Cytotoxicity Assay is a calorimetric alternative to 51Cr release cytotoxicity assays. The CytoTox 96® Assay quantitatively measures lactate dehydrogenase (LDH), a stable cytosolic enzyme that is released upon cell lysis, in much the same way as 51Cr is released in radioactive assays. Released LDH in culture supernatants is measured with a 30-minute coupled enzymatic assay, which results in the conversion of a tetrazolium salt (INT) into a red formazan product. The amount of color formed is proportional to the number of lysed cells. Visible wavelength absorbance data are collected using a standard 96-well plate reader.

### Testing the Effect of FD on Cell Viability by Following Cell Death, Apoptosis and Necrosis Using Flow Cytometry Method

Measurement of cell death by flow cytometry and detection of phosphatidyl serine was performed using the IQ Products kit (R&D systems), according to the manufacturers instructions.

In detail: The Phosphatidyl Serine Detection kit provides a rapid and reliable method for the detection of apoptosis by flow cytometry. This method enables detection at the single-cell level, and also allows the distinction between apoptosis and necrosis.

During the early stages of apoptosis, phosphatidyl serine (PS) becomes exposed on the outside of the cell membrane. This early stage of apoptosis can be specifically detected by PS binding proteins (Annexin V).

During the early stages of apoptosis, the cell membrane is intact and the cells exclude propidium iodide (PI). Later, during the apoptosis process, the membrane becomes porous and PI becomes associated with DNA. The uptake of PI is an indication of necrosis.

### Counting Live and Dead Cells by Trypan Bleu, Using a Standard Microscope

The cells that absorb trypan bleu are dead or in the process of dying.

### Immunophenotypic Staining for Dopamine D1 Receptor and Flow Cytometry Analysis

Normal human T-cells (either resting or following 72 hour TCR-activation) were subjected to single or double immunofluorescence staining, using rabbit antisera directed against either DR1 (Calbiochem) at 1:50 dilution/1×10⁶ cells/100 Al, for 30 minutes on ice. For staining with isotype control, cells were stained with normal rabbit serum (Jackson Immunoresearch Laboratories). The cells were then stained with a fluorescein isothiocyanate (FITC)-conjugated goat anti-rabbit IgG (100 Al of 1:100 dilution; Jackson). In some experiments double staining was performed with PE-conjugated mouse anti-human TCRab mAb (20 Al of stock; Serotec). Cells stained only with the second and third antibodies served as additional negative controls. Fluorescence profiles were recorded in a FACSort.

### Example 2

### Human T-Cell Cancers Express Very High Levels of Dopamine D1R on their Cell Surface, while Normal Human T-Cells do not

The expression of dopamine D1 receptor (D1R) on the cell surface of normal T-cells and cancer T-cell leukemia and lymphoma cells was studied by immunofluorescent staining of these cells, first with rabbit anti-D1R specific antibodies, and then with FITC-conjugated anti-rabbit antibodies, and by flow cytometry analysis using a FACSort. For non-specific isotype control staining, rabbit serum was used.

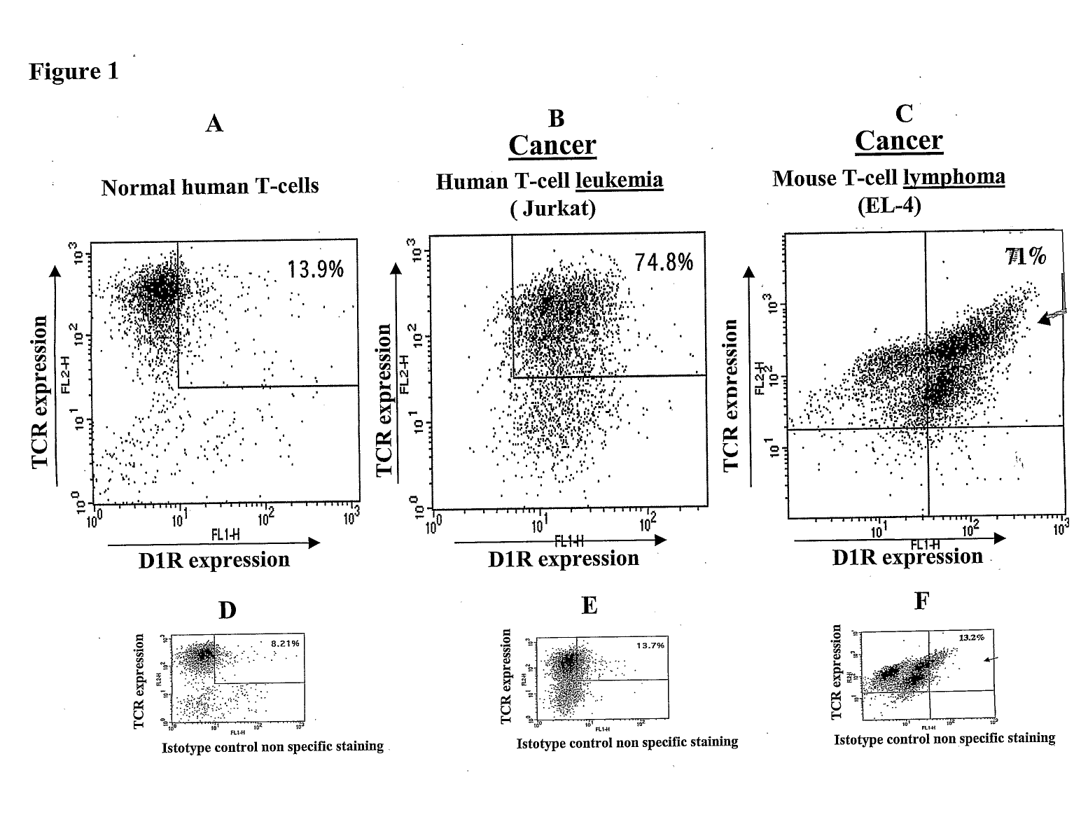

The results, shown in FIG. 1, establish that human T-leukemia cells (Jurkat) and mouse T-lymphoma cells (EL-4) express very high levels of dopamine D1R on their cell surface, while normal human T-cells do not. Thus, the net specific D1R staining on the human leukemia was 61% (74.8% specific staining-13.7% control non-specific staining), on human T-lymphoma 57.8% (71% specific staining-13.2% control non-specific staining), while on normal peripheral human T-cells only 5.7% (13.9% specific staining-8.2% control non-specific staining) (FIG. 1A-F).

It was further found that several types of non-T human lymphoma and leukemia, i.e., human Burkitt's B-lymphoma (Daudi and Raji) and human Chronic-Myeloid Leukemia (CML) (K-562) cells also express various extents of the D1R on their cell surface (data not shown).

### Example 3

### Fenoldopam Mesylate Kills Human Cancer Leukemia and Lymphoma, Evident by the Number of Surviving Cells

Further tests were conducted to establish that selective D1R agonists, such as fenoldopam mesylate (FDM), which is also an FDA-approved drug for regulating blood pressure, can kill human cancer cells expressing the dopamine D1R. For this purpose, the Jurkat T-cell leukemia line, the HuT-78 human T-lymphoma (cutaneous “Sezary” T-lymphoma) line, and the K-562 human Chronic-Myeloid Leukemia (CML) and Daudi Human B-lymphoma (Burkitt's lymphoma) lines were seeded in tissue culture wells (0.5 million cells/0.5 ml/well). FDM (from the original clinically used ampoule, original concentration, MW=401, 10 mg/ml=25 mM) was diluted with 0.9% sodium chloride injection (as instructed by the manufacturer) to serial dilutions of 10⁻² M-10⁻¹⁰ M. Then, FDM was added to the corresponding microtiter wells (5 microliter of FDM at a give concentration to 0.5 ml cells, dilution of 1:100), so that the final FDM concentrations tested were 10⁻⁴M-10⁻¹² M.

FDM (at each of the above mentioned concentrations) was added to the corresponding microtiter well four times during 1 hour total, at time 0, 15 minutes, 30 minutes and 60 minutes. Cell survival/death was evaluated 3 days later by counting the number of living cells, using flow cytometry.

Table 1 shows that FDM killed the human T-cell leukemia, Sezary T-cell lymphoma and chronic myeloid leukemia (CML) in a very significant and dose dependent manner.

TABLE 1

| | Human T-Cell | | Human Sezary Cell | | | |
|---|---|---|---|---|---|---|
| | Leukemia (Jurkat) | | Lymphoma (HuT-78) | | Human CML (K562) | |
| | No. of | | No. of | | No. of | |
| | Living | % Dead | Living | % Dead | Living | % Dead |
| | Cells | Cells | Cells | Cells | Cells | Cells |
| Untreated | 19419 | | 23422 | | 28314 | |
| FDM 10⁻⁴M | 4 | ~100% | 213 | 99% | 21 | 100% |
| FDM 10⁻⁶M | 15938 | 18% | 17023 | 28% | 18680 | 34% |
| FDM 10⁻⁸M | 7451 | 62% | 16080 | 32% | 21137 | 26% |
| FDM 10⁻¹⁰M | 9472 | 52% | 14728 | 38% | 21476 | 25% |
| FDM 10⁻¹²M | 10568 | 46% | 12662 | 46% | 19250 | 33% |

Interestingly, Table 1 shows that 1 hour of 10⁻⁴ M FDM (the original FDM concentration injected to patients for FDA-approved 48 hour infusion treatment for reducing their blood pressure) causes the killing of all the cancer cells. A 10,000 lower concentration of 10⁻⁸ M (=0.1 nM) FDM, which is the reported approximate steady state concentration of FDM in the circulation of patients receiving the 48 hour FDA-approved infusion, caused the death of 62% of the human T-leukemia, 32% of the human Sezary T-lymphoma and 25% of the human CML.

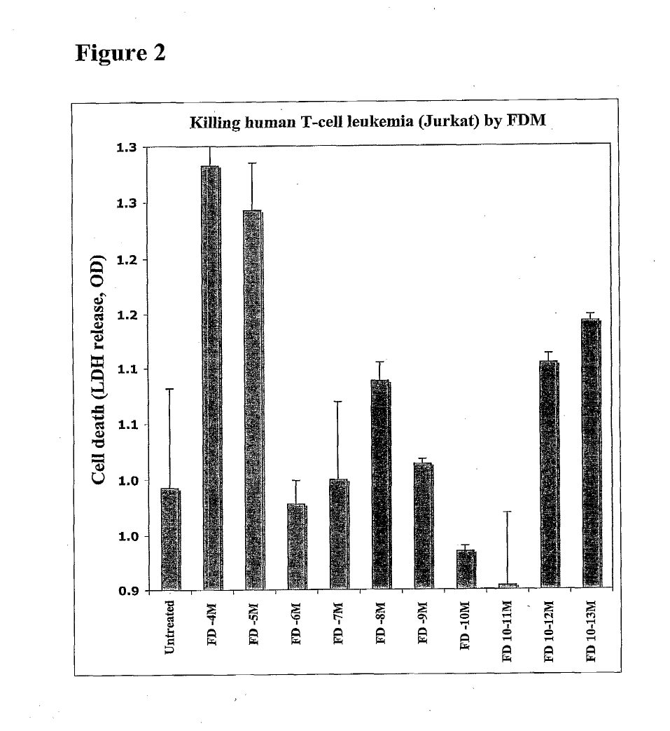

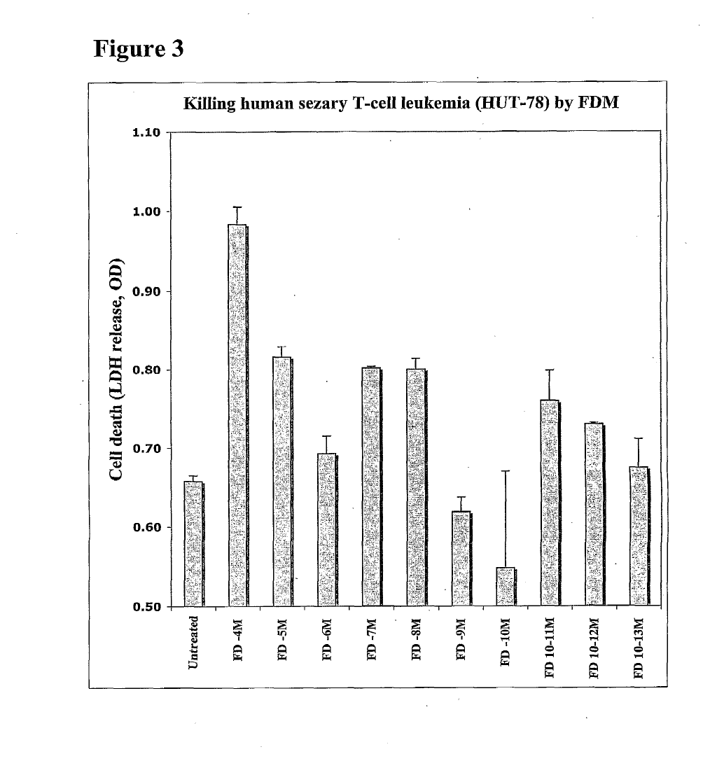

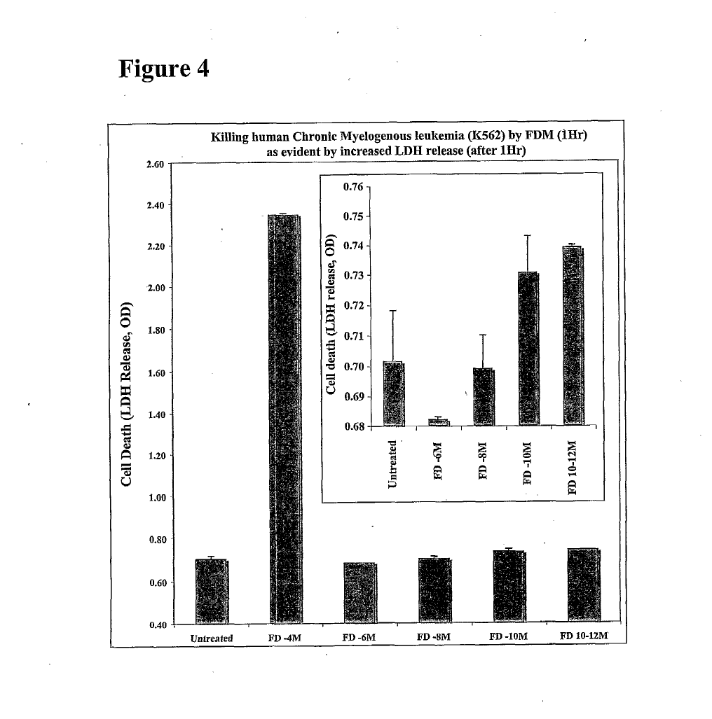

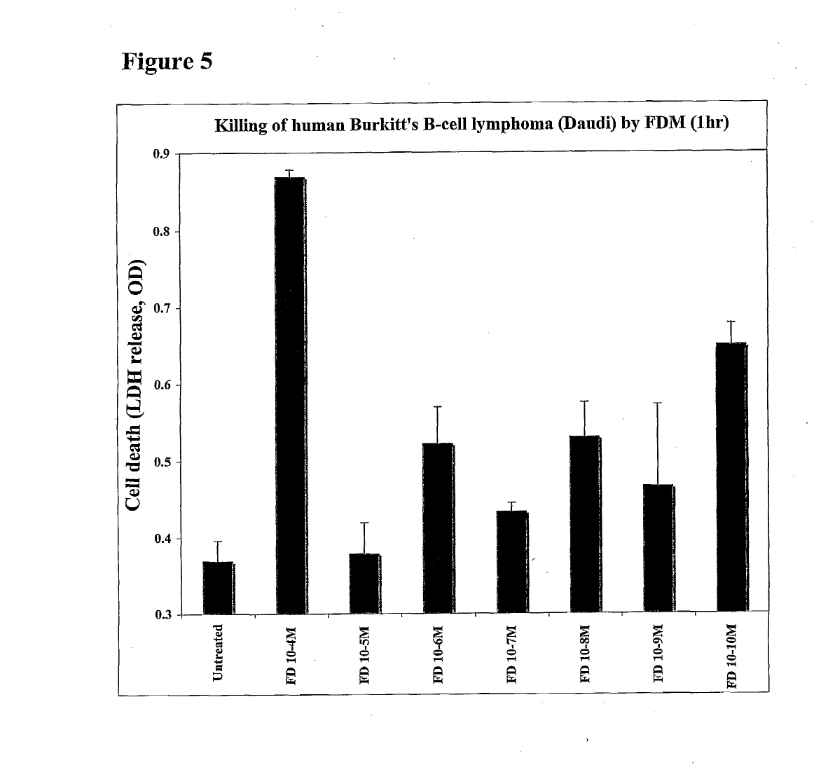

In subsequent experiments, using the same human T-leukemia, T-lymphoma and CML cells mentioned above as well as human Burkitt's B cell lymphoma (Daudi), it was shown that FDM at several concentrations (once again added to the cells four times, 15 minutes apart, during a total of 1 hour), killed cells of all four types of human T-cell, B-cell and CML cancers as evident by the augmented release of lactate dehydrogenase (LDH), a stable cytosolic enzyme that is released upon cell death/lysis (FIGS. 2-5).

Of note, the augmented LDH release was measured immediately after the 1 hour of FDM addition. Despite the clear killing effect of FDM, dose-dependency of this effect was complex, unexpected and different to each of the cancer types (FIGS. 2-5).

### Example 4

### Activated Normal Human T-Cells Also Express Very High Levels of Dopamine D1R on their Cell Surface, while Resting Normal Human T-Cells do not

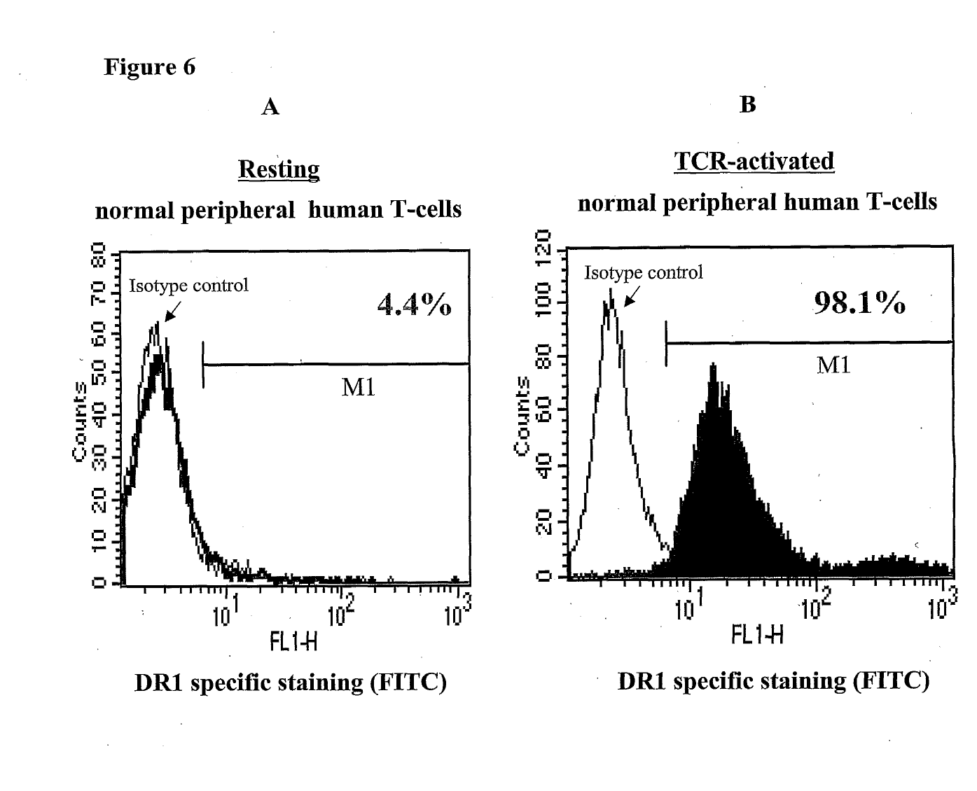

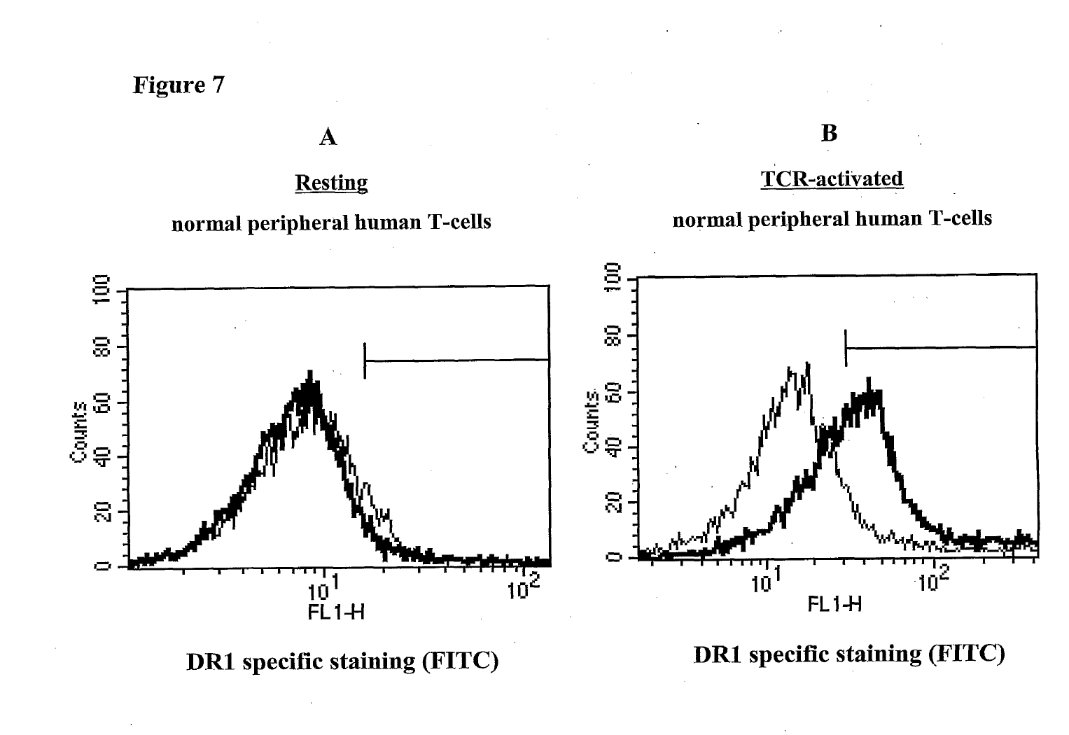

The dopamine D1R is also expressed in very high levels in normal (i.e., non-cancer) peripheral human T-cells that underwent “classical” T-cell receptor (TCR) activation in vitro (using anti-CD3 and anti-CD28 monoclonal antibodies), while “resting” (i.e., not activated) normal human T-cells do not (FIGS. 6 and 7, representing T-cell derived from two different healthy human individuals). Such TCR-activation is commonly used to mimic the in vivo situation whereby T-cells, which encounter foreign antigens presented by appropriate antigen presenting cells (APC's), become highly activated via the TCR.

### Example 5

### Fenoldopam Mesylate Induces Marked Death of TCR-Activated Normal Human Peripheral T-Cells, but not Resting Normal Human T-Cells

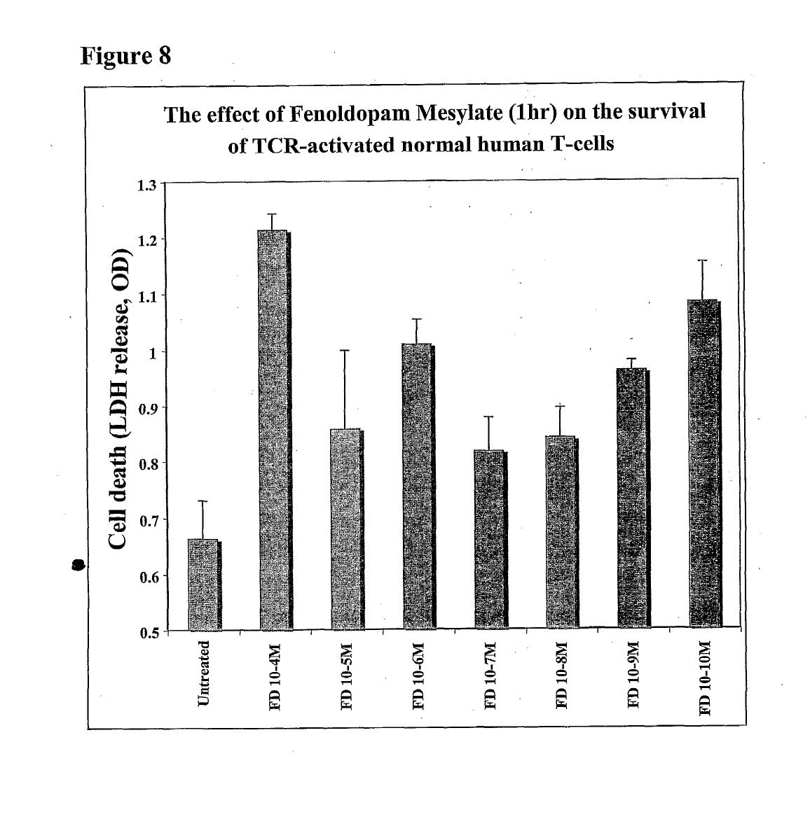

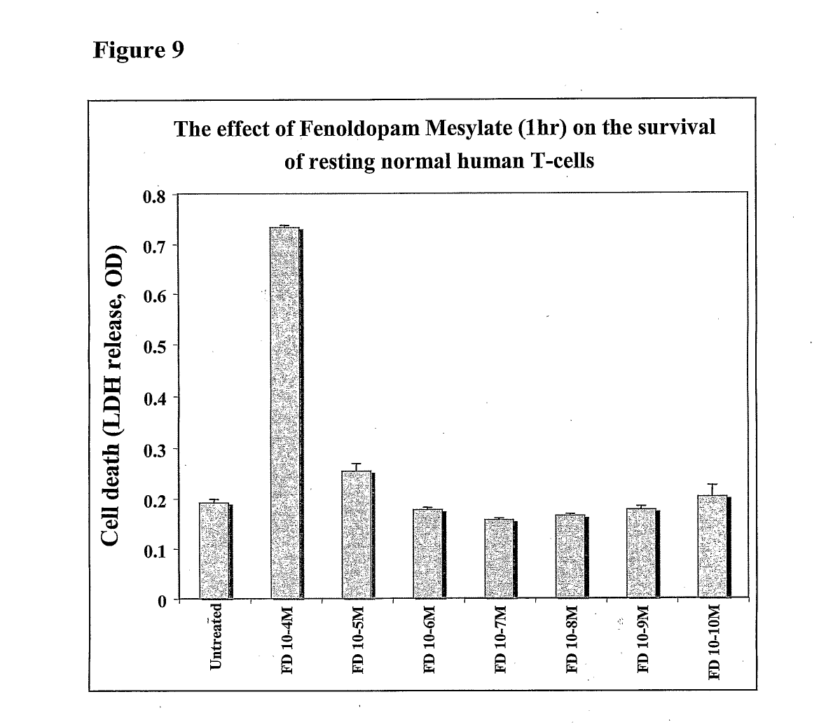

In line with the elevated levels of D1R expression found herein in TCR-activated normal human T-cells (FIGS. 6 and 7), FDM, at 10⁻⁴ M-10⁻¹⁰ M, caused a marked death of these activated cells (FIG. 8), while hardly affecting the resting normal human T-cells (FIG. 9); the latter resting cells were in fact killed only by the highest FDM concentration tested herein (10⁻⁴ M).

### Example 6

### Effect of Fenoldopam Hydrobromide on Human Leukemia and Lymphoma

Next, fenoldopam hydrobromide, which has similar chemical structure to FDM, was tested for its ability to kill human leukemia and lymphoma. Tables 2-4 show that this is indeed the case, as fenoldopam hydrobromide, in a dose and time-dependent manner, increased substantially the release of LDH from the human B-cell lymphoma (Table 2), T-cell lymphoma (Table 3) and CML (Table 4). Table 2 shows that the maximal killing of the human B-cell lymphoma was observed with 10⁻⁸ M fenoldopam hydrobromide.

Tables 3 and 4 show results of experiments designed primarily for studying the kinetics of the effect (herein fenoldopam hydrobromide was tested only at a concentration range of 10⁻⁴ M-10⁻⁶ M), and indicate that already after 1 minute of fenoldopam hydrobromide addition, there is an increased LDH. Yet, the extent of death increased gradually with time (10, 30 and 60 minutes), and after 1 hour the cancer cells released dramatic levels of LDH, indicating massive cell death.

TABLE 2
Fenoldopam (1 hour) Kills Human Burkitt's
B-Cell Lymphoma (Daudi)

| | LDH Release (OD) | | |
|---|---|---|---|
| | OD Duplicates | Average | STDEVP |
| Untreated | 0.581 | 0.5735 | 0.0075 |
| | 0.566 | | |
| +Fenoldopam 10⁻⁴M | 0.958 | 0.9595 | 0.0015 |
| | 0.961 | | |
| +Fenoldopam 10⁻⁵M | 0.99 | 1.0025 | 0.0125 |
| | 1.015 | | |
| +Fenoldopam 10⁻⁶M | 0.525 | 0.526 | 0.001 |
| | 0.527 | | |
| +Fenoldopam 10⁻⁷M | 0.55 | 0.5365 | 0.0135 |
| | 0.523 | | |
| +Fenoldopam 10⁻⁸M | 1.316 | 1.339 | 0.023 |
| | 1.362 | | |
| +Fenoldopam 10⁻⁹M | 0.903 | 0.899 | 0.004 |
| | 0.895 | | |

TABLE 3
Fenoldopam Kills Human T-Cell Lymphoma (HuT-78)

| | Release of LDH (OD) | | |
|---|---|---|---|
| | OD | | |
| | Duplicates | Average OD | STDEVP |
| Untreated | 0.599 | 0.594 | 0.005 |
| | 0.589 | | |
| +Fenoldopam 10⁻⁴M 1 Min | 0.841 | 0.8135 | 0.0275 |
| | 0.786 | | |
| +Fenoldopam 10⁻⁴M 10 Min | 2.124 | 1.4355 | 0.6885 |
| | 0.747 | | |
| +Fenoldopam 10⁻⁴M 30 Min | 3.015 | 3.087 | 0.072 |
| | 3.159 | | |
| +Fenoldopam 10⁻⁴M 60 Min | 2.688 | 2.8775 | 0.1895 |
| | 3.067 | | |
| Untreated | 0.599 | 0.594 | 0.005 |
| | 0.589 | | |
| +Fenoldopam 10⁻⁴M 60 Min | 2.688 | 2.8775 | 0.1895 |
| | 3.067 | | |
| +Fenoldopam 10⁻⁵M 60 Min | 2.907 | 2.9465 | 0.0575 |
| | 3.022 | | |
| +Fenoldopam 10⁻⁶M 60 Min | 0.759 | 0.7625 | 0.0035 |
| | 0.766 | | |

TABLE 4
Fenoldopam Kills Human Chronic
Myeloid Leukemia (CML, K-562)

| | Release of LDH (OD) | | |
|---|---|---|---|
| | OD | | |
| | Duplicates | Average OD | STDEVP |
| Untreated | 0.82 | 0.819 | 0.001 |
| | 0.818 | | |
| +Fenoldopam 10⁻⁴M 1 Min | 1.081 | 1.081 | 0 |
| +Fenoldopam 10⁻⁴M 10 Min | 0.868 | 0.863 | 0.005 |
| | 0.858 | | |
| +Fenoldopam 10⁻⁴M 30 Min | 2.737 | 2.7755 | 0.0385 |
| | 2.814 | | |
| +Fenoldopam 10⁻⁴M 60 Min | 2.733 | 2.27 | 0.463 |
| | 1.807 | | |
| Untreated | 0.82 | 0.819 | 0.001 |
| | 0.818 | | |
| +Fenoldopam 10⁻⁴M 60 Min | 2.733 | 2.27 | 0.463 |
| | 1.807 | | |
| +Fenoldopam 10⁻⁵M 60 Min | 2.907 | 2.9645 | 0.0575 |
| | 3.022 | | |
| +Fenoldopam 10⁻⁶M 60 Min | 0.759 | 0.7625 | 0.0035 |
| | 0.766 | | |

### Example 7

### Effect of Other Selective Dopamine D1R Agonists on Lymphoma and Leukemia Cells

Three additional highly selective dopamine D1R agonists were also shown to kill human lymphoma and leukemia cells. These highly selective D1R agonists included the A 77636 hydrochloride, referred to as “potent, selective D1-like agonist, orally active;” SKF 38393 hydrobromide, referred to as “D1-like dopamine receptor selective partial agonist;” and A 68930 hydrochloride, referred to as “potent and selective D1-like dopamine receptor agonist” (Tocris Cookson Catalogue).

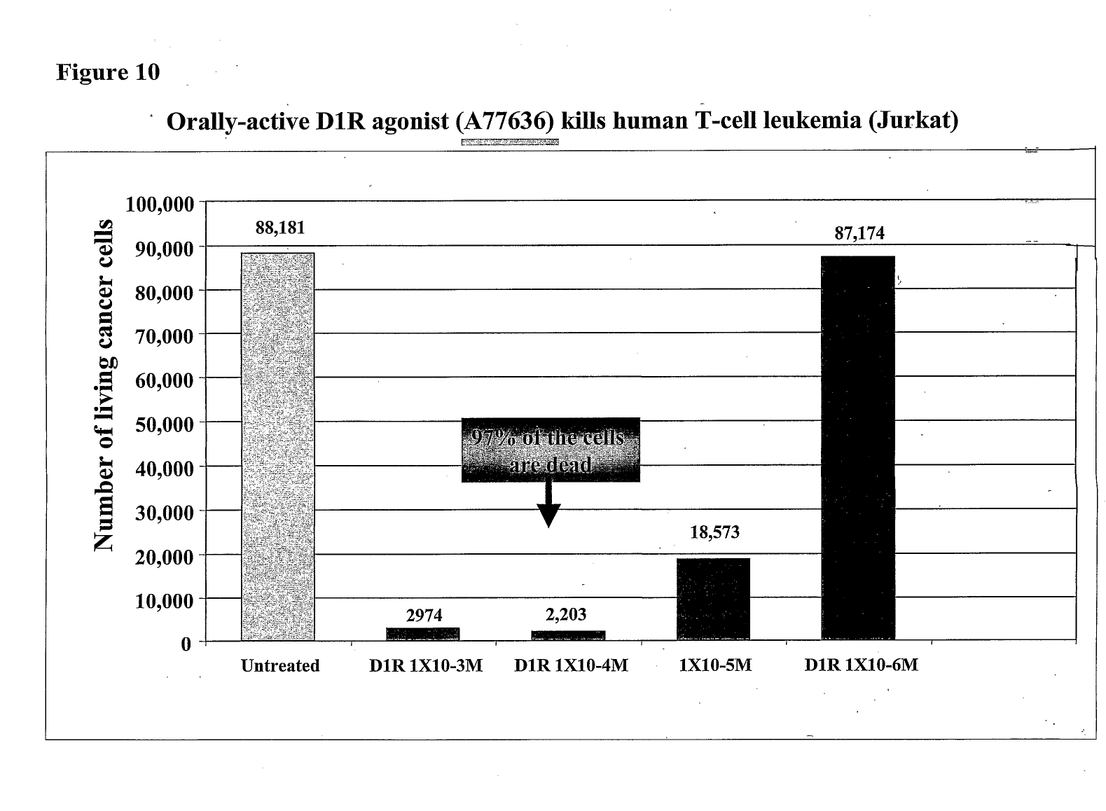

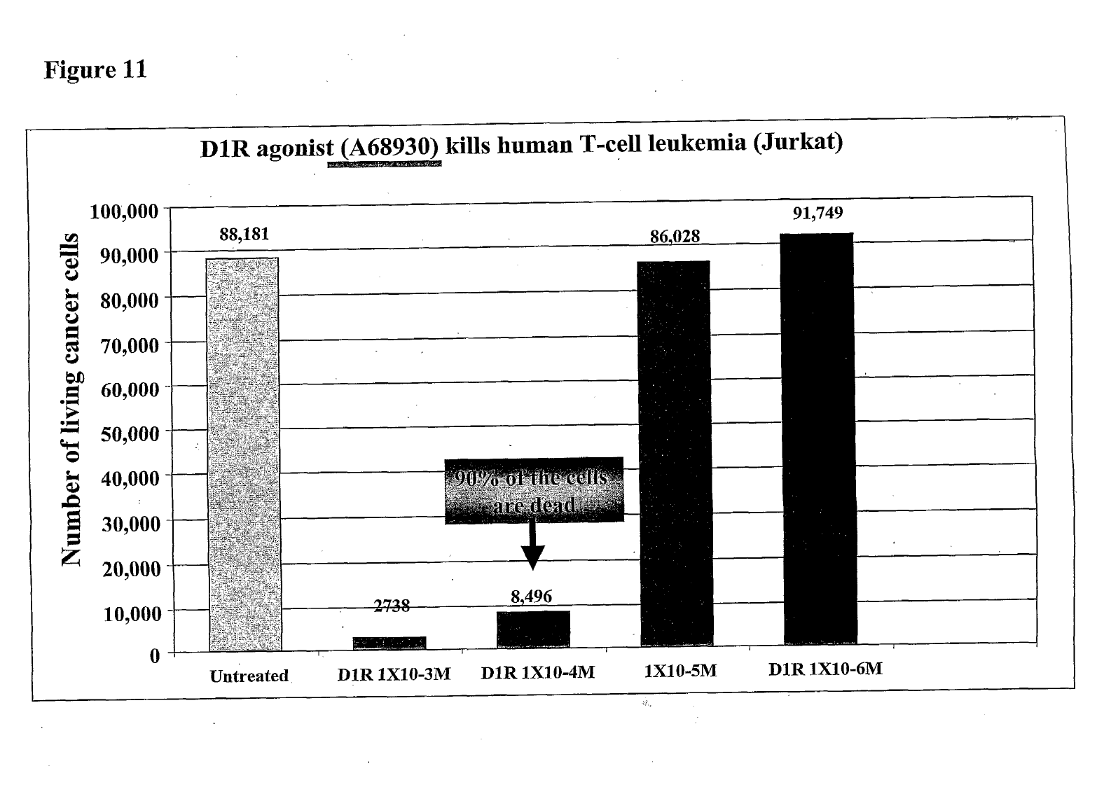

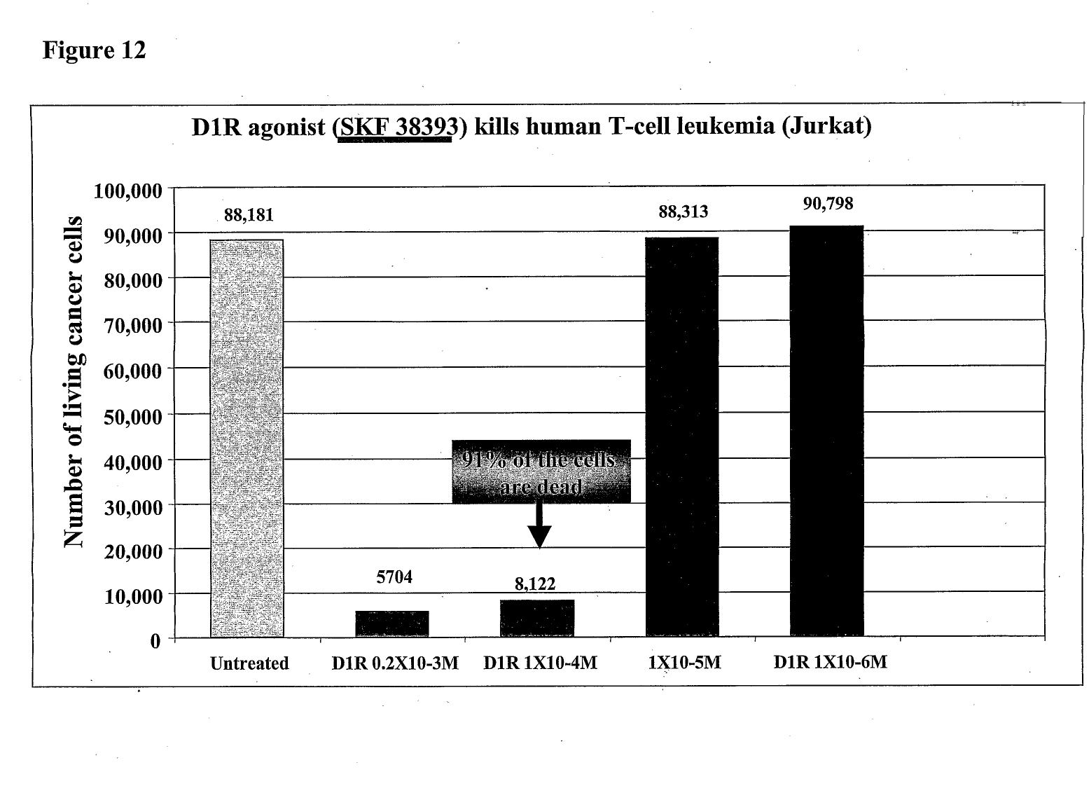

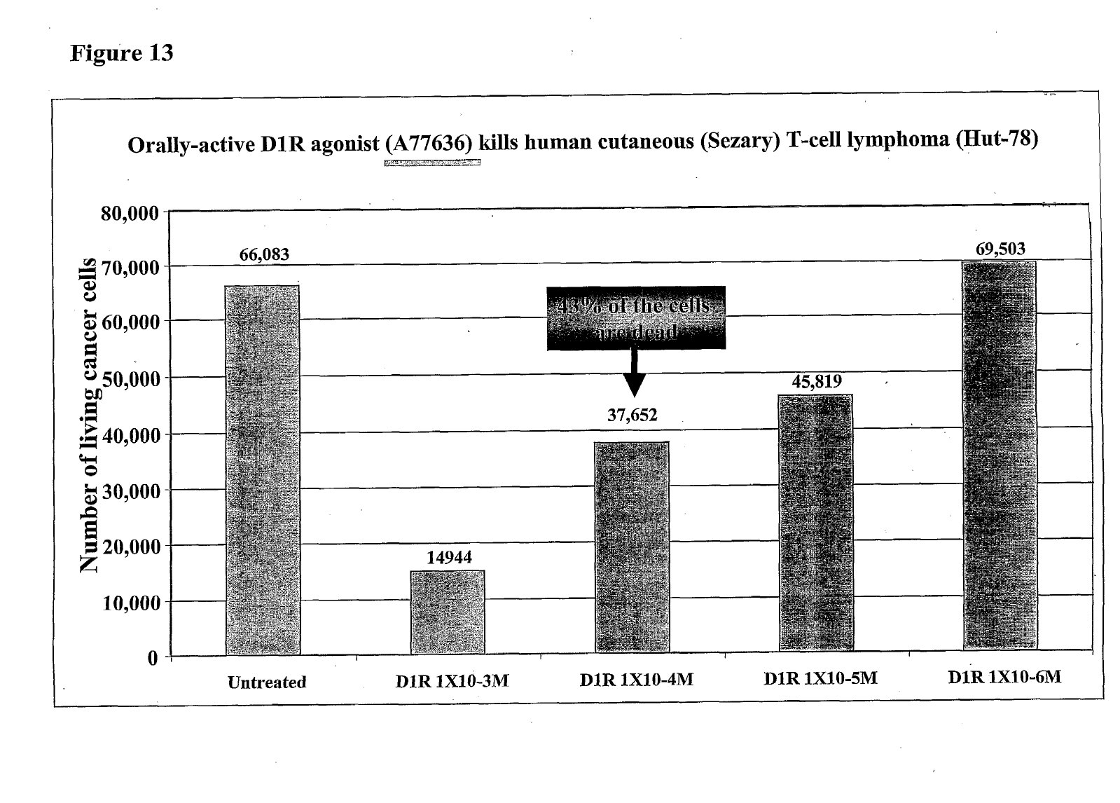

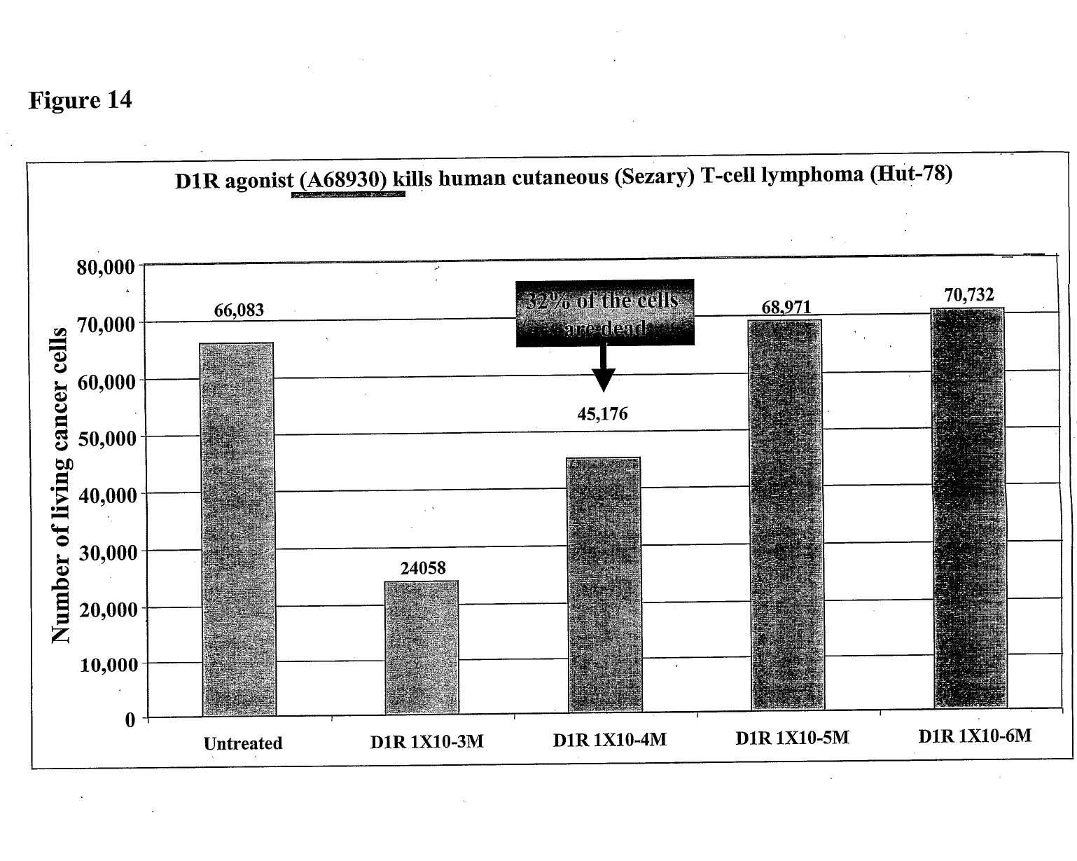

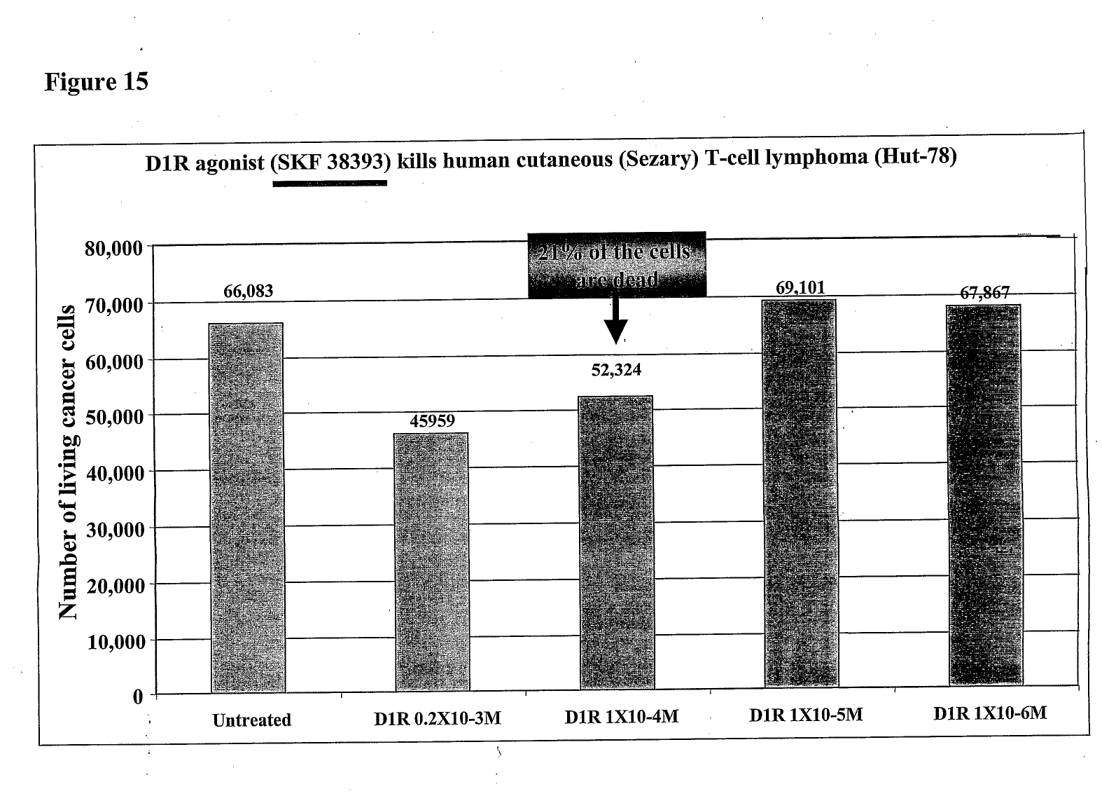

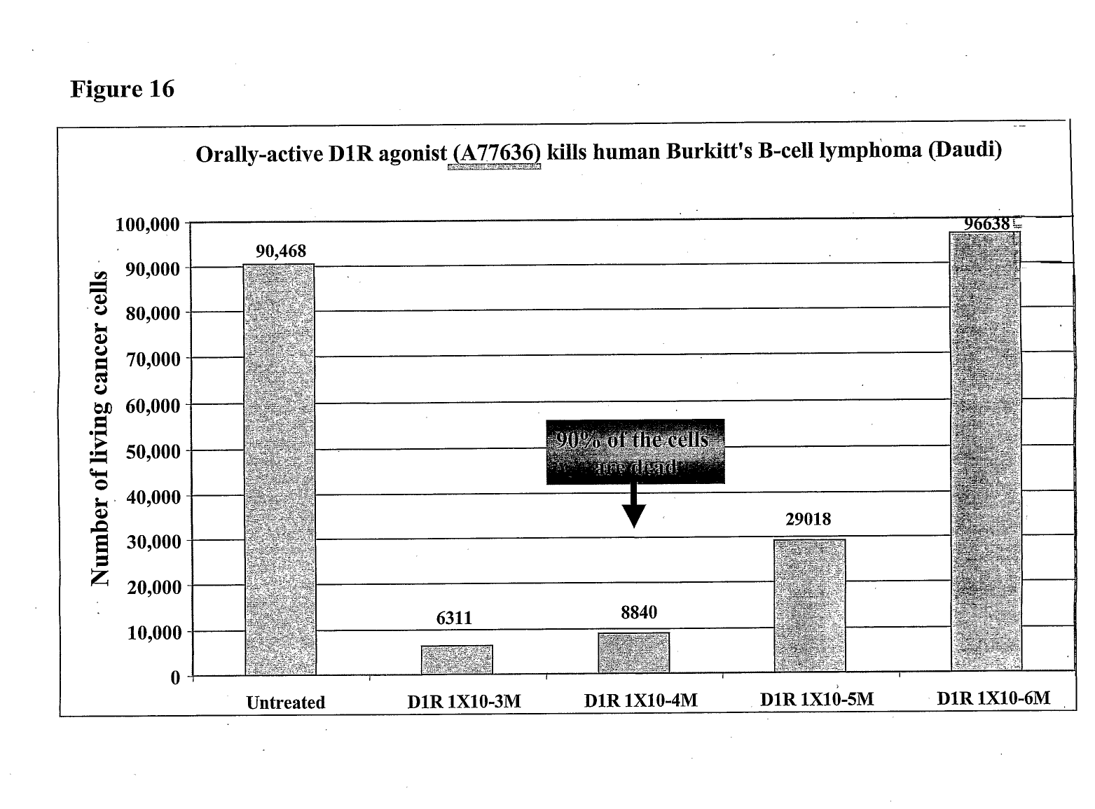

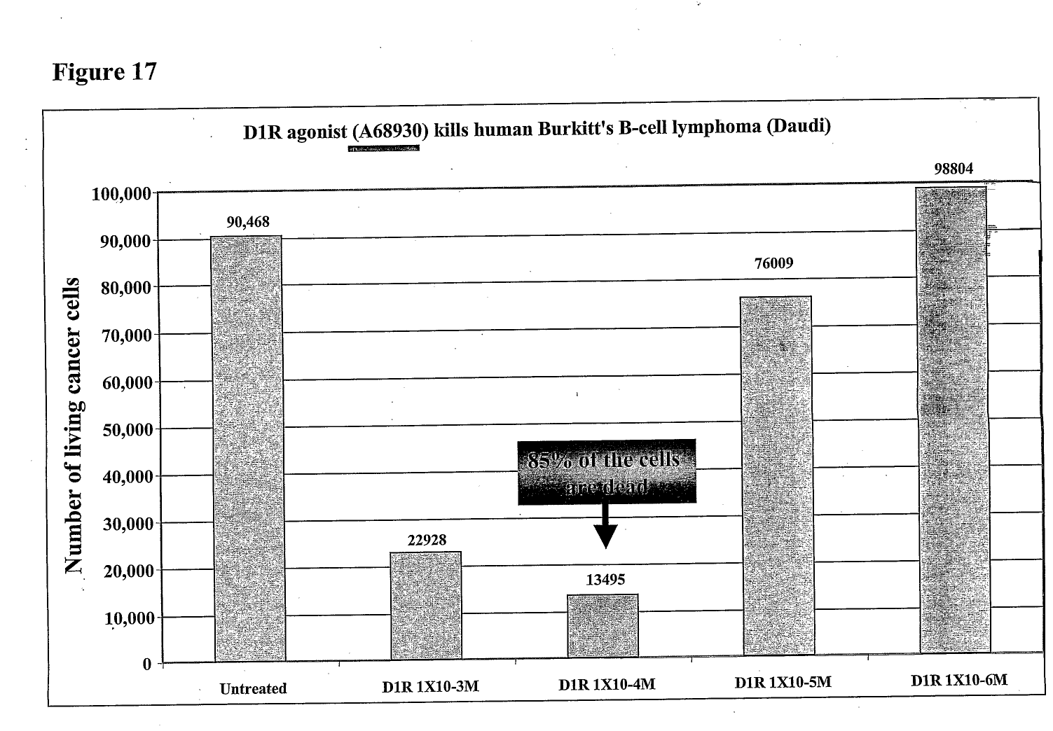

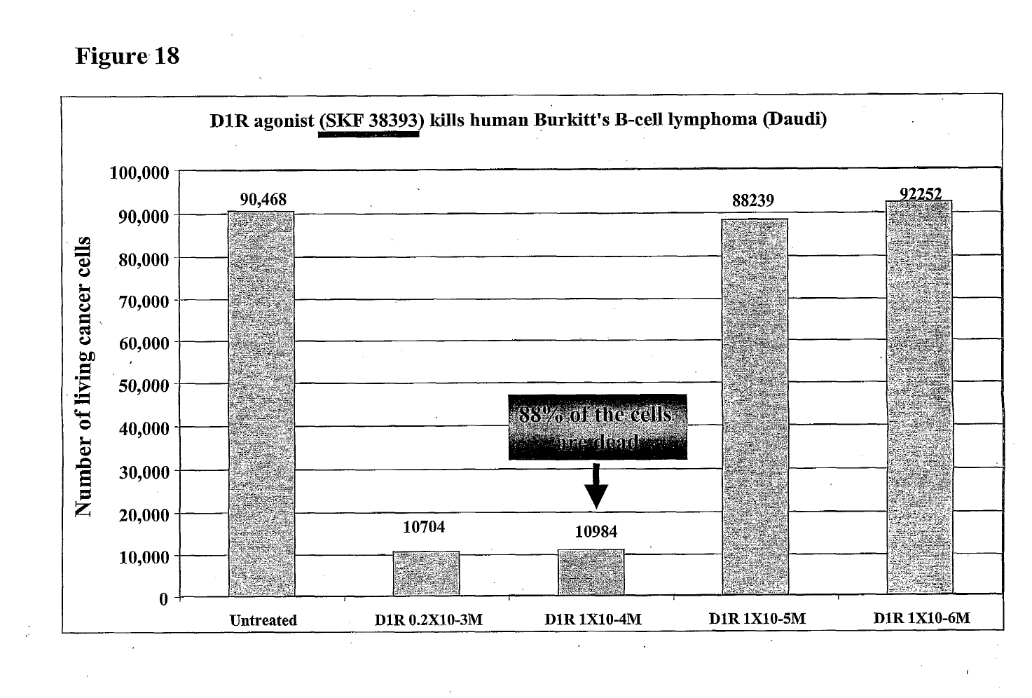

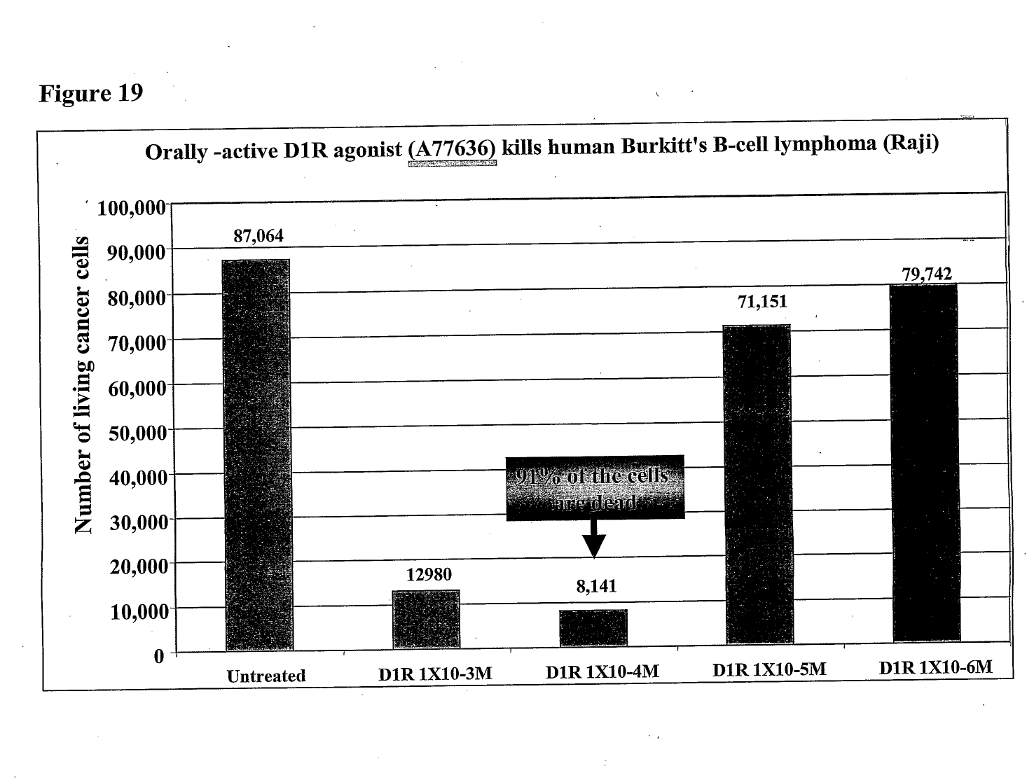

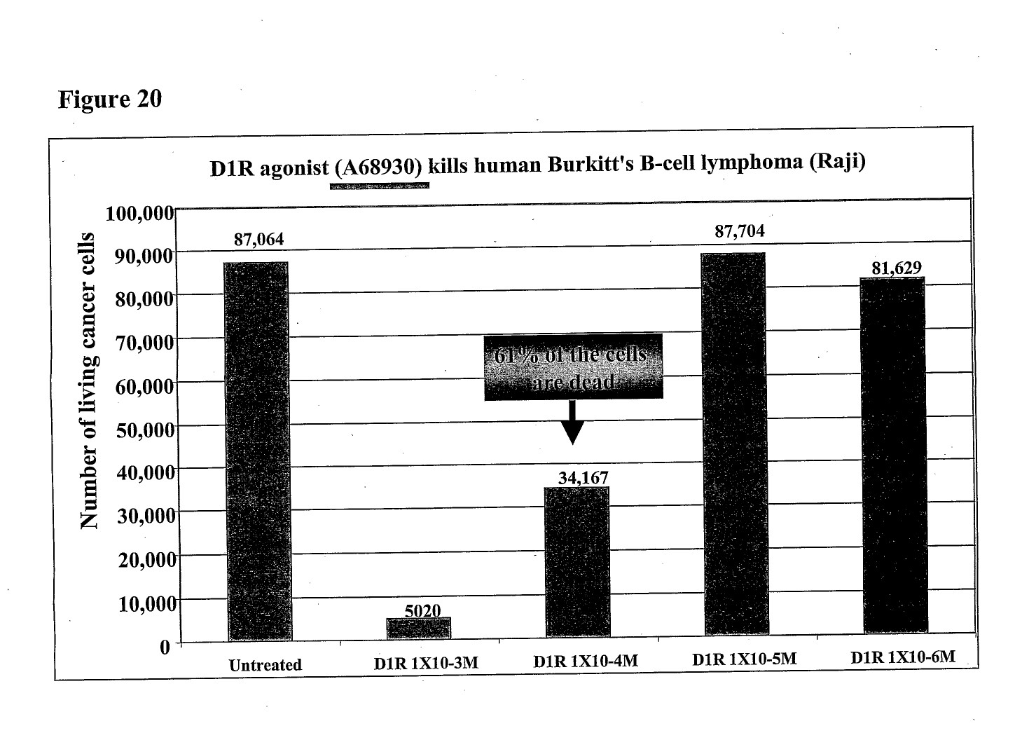

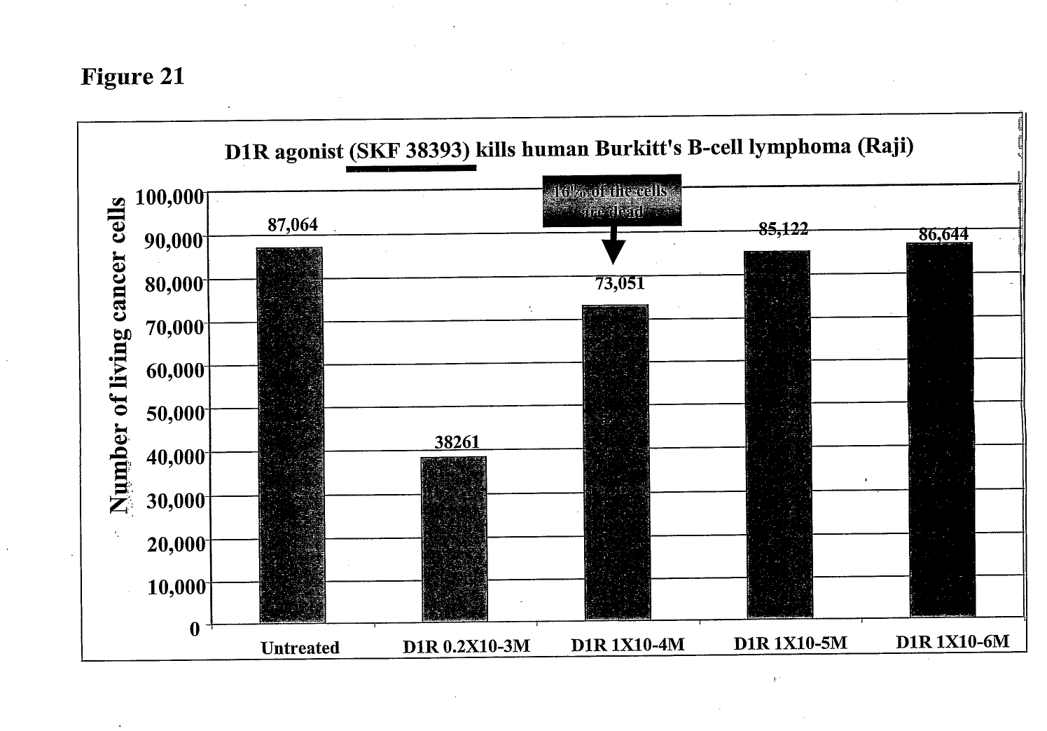

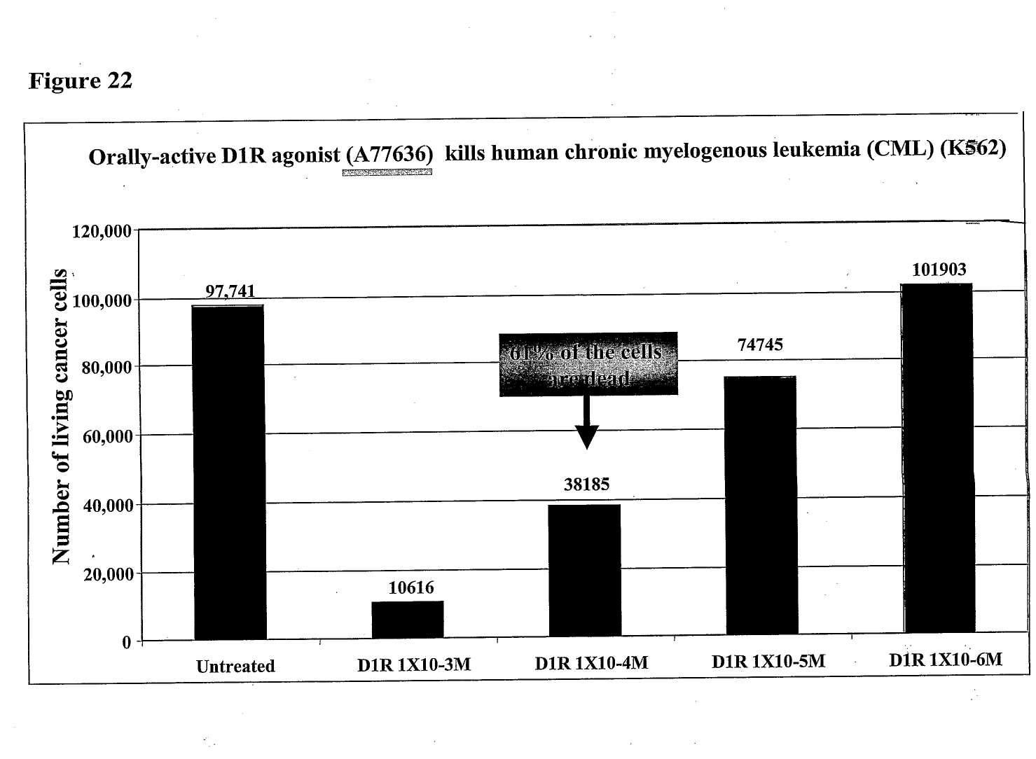

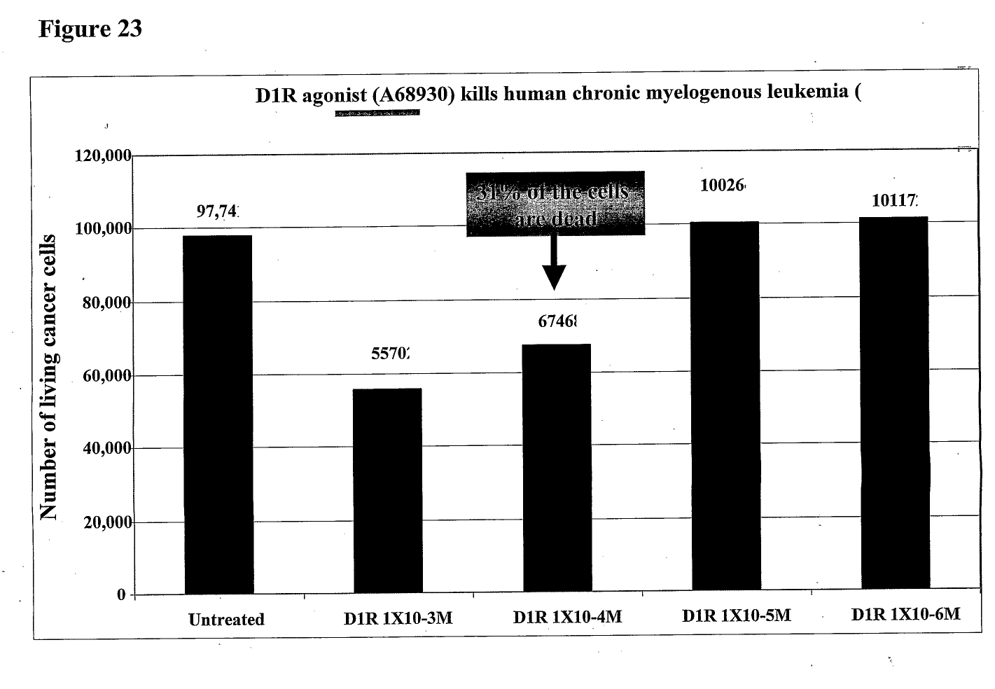

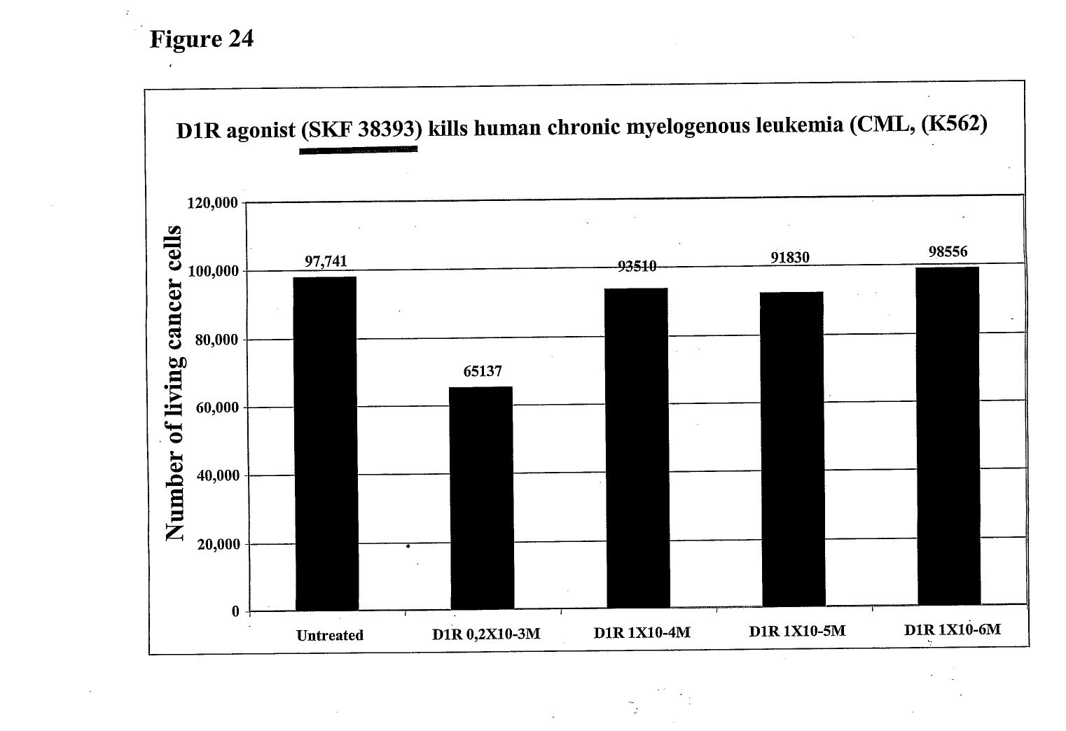

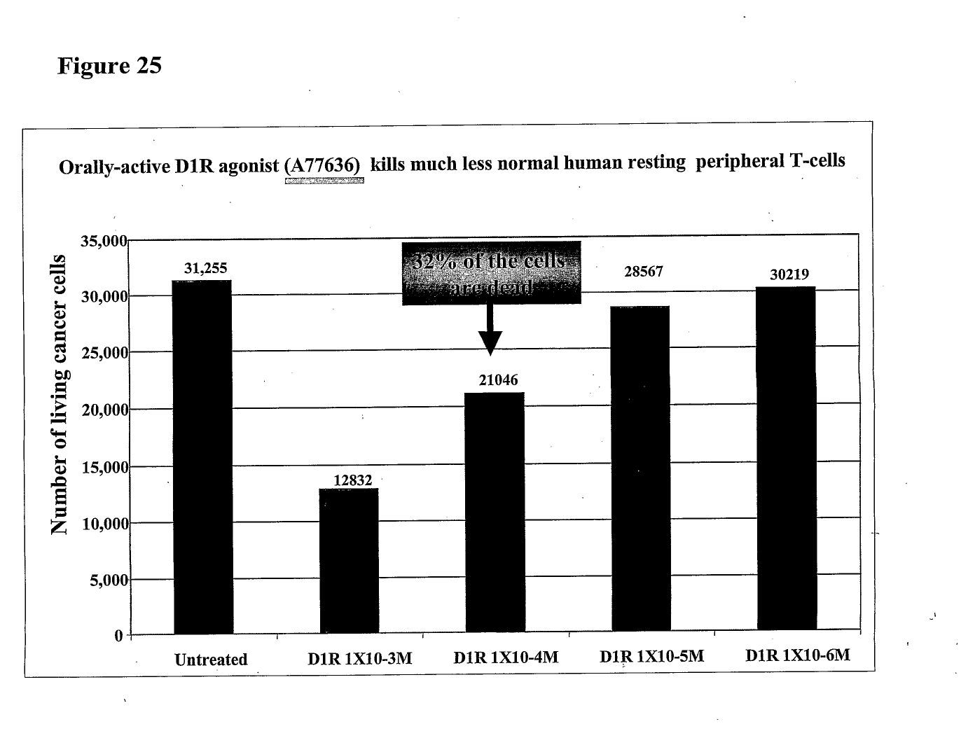

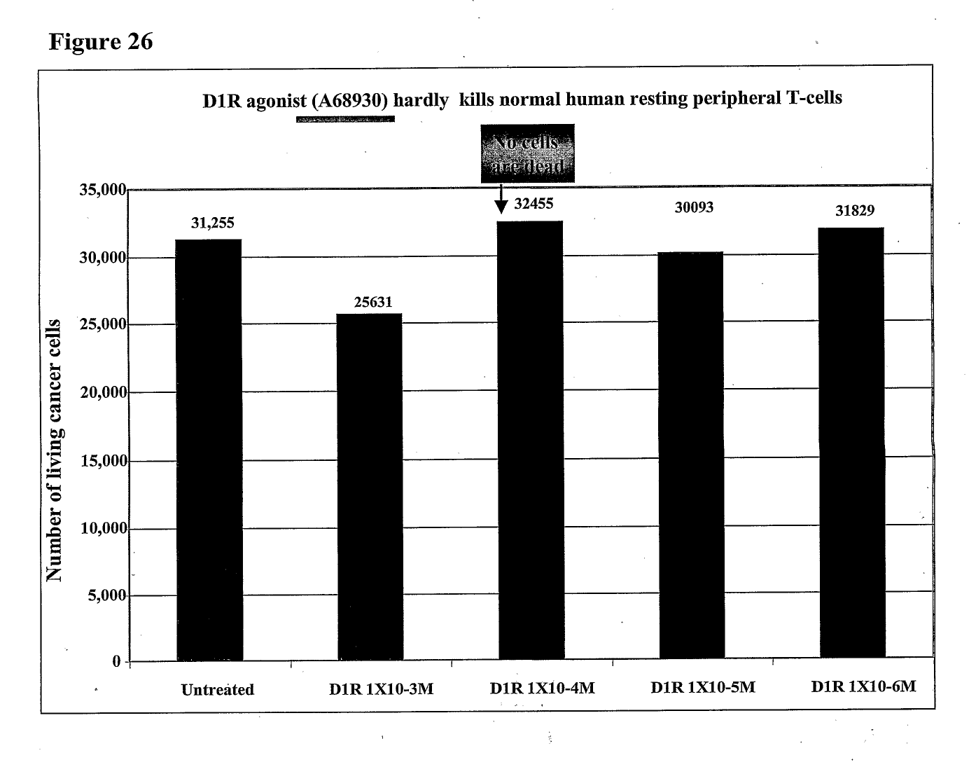

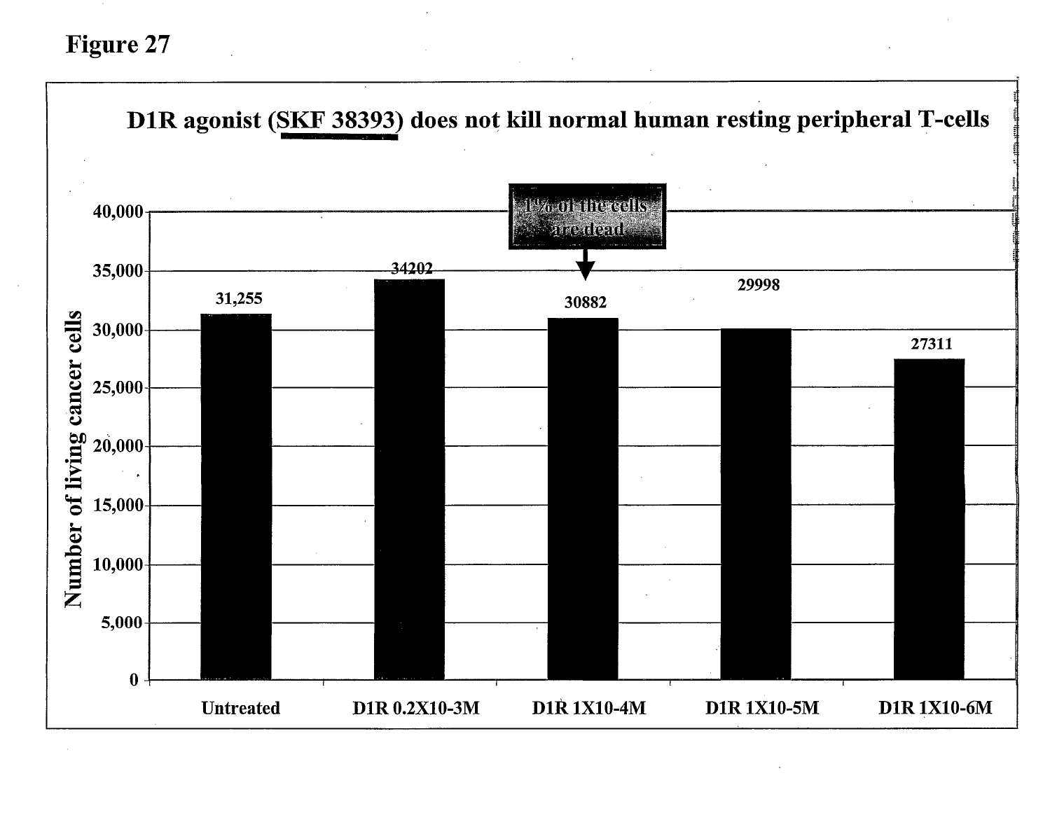

These three highly selective D1R agonists indeed killed, in a dose-dependent manner, substantial numbers of human T-cell leukemia (FIGS. 10-12), T-cell lymphoma (FIGS. 13-15), two types of B-cell lymphoma (FIGS. 16-18: Daudi; FIGS. 19-21: Raji)), and CML (FIGS. 22-24). In contrast, these D1R agonists had a substantially lower effect, if at all, on normal (i.e., non-cancer) human T-cells (FIGS. 25-27). In all the above set of experiments (FIGS. 10-24), cell death was evaluated by the number of surviving cells 3 days after addition of the D1R agonists. Interestingly, the three D1R agonists differed in regards to their killing potencies, the most effective usually being the A 77636 hydrochloride. Furthermore, the extent of cancer cell death induced by a given D1R agonist varied from one cancer type of cancer to the other (FIGS. 10-24).

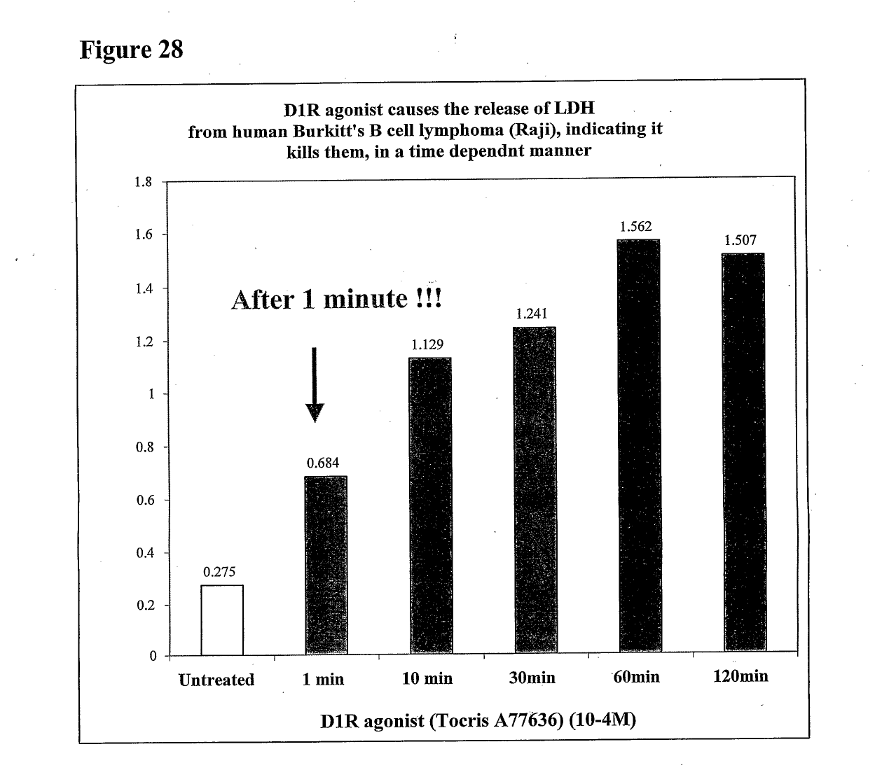

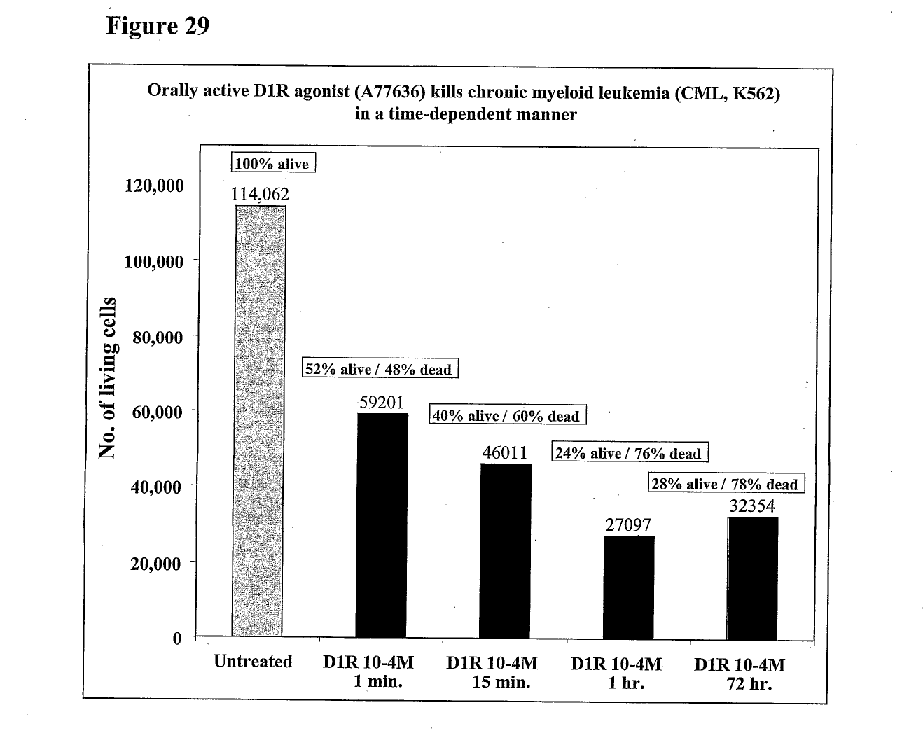

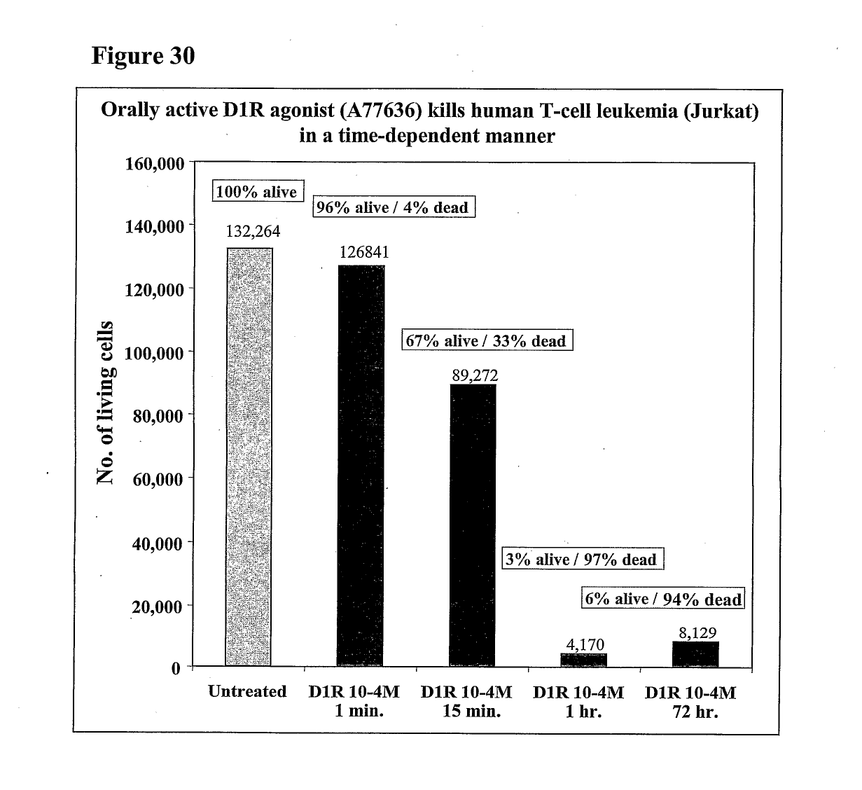

Cancer death induced by selective D1R agonists is highly specific to the D1 receptor. FIG. 28 shows that exposure of human B cell cancer for 1 minute only to a D1R agonist (in this case the A77636) is sufficient to kill the cells, as evident by a ≈3 fold elevation in the release of LDH. A longer exposure to LDH (for 10, 30, and 60 minutes) caused a further increase in the extent of cell death, reaching a plateau at 1 hour so that adding of the D1R agonist for 2 hours was not significantly more effective. FIG. 29 shows CML exposure for 1 minute only to a D1R agonist (and then washing the cells and resuspension in D1R-agonist free medium) was sufficient to kill ≈48% of the cells, as evident from the number of living cells counted by flow cytometry 3 days later. Exposure of the CML cells to 15 minutes or 1 hour of D1R agonist killed 60% and 76% of the cells respectively. Much longer incubations of the CML cells with the D1R agonist (72 hours) had no additional value beyond the 1-hour effect. FIG. 30 shows that for the T-leukemia cells, 1 min incubation with the D1R agonists was not sufficient to cause marked cell death. The effect becomes significant after 15 minutes, and reached a maximum-killing of 94% of the cells, after 1 hour of incubation. Once again, 72 hour incubation with the D1R agonists had no further effect.

Cancer Death is Induced Only by Selective D1R Agonists, and not by D2R and D3R Agonists, Showing that the Effect was Mediated Specifically by the D1R Receptor.

To test the selectivity of the effect induced by dopamine D1R agonists, the effects of highly selective agonists for the dopamine D2R-Quinpirole, and D3R-R7-Hydroxy-DPAT, were tested in parallel (i.e., within the same experiments). The effect of dopamine itself (that can of course activate all its D1R-5 receptors) was also tested. All of these molecules were tested at a similar concentration (10⁻⁴ M). Tables 5 and 6 show that while the D1R agonist (1 hour) killed a substantial number of human B-lymphoma and CML, the D2R and D3R agonists had no such effect. The specificity and restriction of the effect to the D1R is also seen in Tables 7 and 8. These results show that the killing of the cancer cells was mediated specifically by the dopamine D1R. Interestingly, dopamine itself killed the B-lymphoma cells but not the CML (Tables 5 and 6).

TABLE 5
Only a Dopamine D1R Agonist (or Dopamine Itself) But Not
D2R or D3R Agonists Kill Human B Cell Lymphoma (Daudi)

| | No. of Cells |
|---|---|
| Untreated | 21,225 |
| D1R Agonist 1 × 10⁻⁴M | 4,680 |
| D2R Agonist 1 × 10⁻⁴M | 20,805 |
| D3R Agonist 1 × 10⁻⁴M | 18,330 |
| Dopamine | 5,325 |

TABLE 6
Only a Dopamine D1R Agonist But Neither D2R
or D3R Agonists Nor Dopamine Itself Kill Human
Chronic Myeloid Leukemia Cells (CML K562)

| | No. of Cells |
|---|---|
| Untreated | 22,485 |
| D1R Agonist 1 × 10⁻⁴M | 13,500 |
| D2R Agonist 1 × 10⁻⁴M | 27,360 |
| D3R Agonist 1 × 10⁻⁴M | 21,960 |
| Dopamine | 24,480 |

### Example 8

### Study of Mechanism of Cell Death After Incubation with DR1Agonists

Cancer death induced by selective D1R agonists occurs via necrosis. To study the mechanism by which the cancer cells die, due to their incubation with DR1 agonists, the phosphatidyl serine detection kit was used. This kit provides a rapid and reliable method for the detection of apoptosis by flow cytometry, enables detection at the single-cell level, and also allows the distinction between apoptosis and necrosis.

During the early stages of apoptosis, phosphatidyl serine (PS) becomes exposed on the outside of the cell membrane. This early stage of apoptosis can be specifically detected by PS binding proteins (Annexin V). During the early stages of apoptosis, the cell membrane is intact and the cells exclude propidium iodide (PI). Later, during the apoptosis process, the membrane becomes porous and PI becomes associated with DNA. The uptake of PI is an indication of necrosis. Thus, Annexin V⁺ PI⁻ are considered cells that are undergoing apoptosis, while Annexin V⁺ PI⁺ are considered cells that are undergoing necrosis. Live cells are Annexin V⁻ PI⁻.

Tables 7 and 8 show that the T-leukemia and T-lymphoma cells, which are exposed for 1 hr to a D1R (but not D2R or D3R) agonist, die primarily via a mechanism of necrosis. Indeed, after 1 hour the percent of Annexin V⁺ PI⁺ necrotic T-leukemia cells raised dramatically from 6.3% to 90.4%, in parallel to a marked reduction in the number of living cells, while the percent of apoptotic cells did not change (Table 7).

TABLE 7
Killing Human T-Cell Leukemia (Jurat) Cells
by D1R, D2R or D3R Agonists or Dopamine

| | No. of | % Necrotic | % Apoptotic |
|---|---|---|---|
| | Cells | Cells | Cells |
| Untreated | 12,990 | 6.30% | 1.40 |
| +D1R Agonist 1 × 10⁻⁴M | 2,115 | 90.40% | 1.23 |
| +D2R Agonist 1 × 10⁻⁴M | 7,395 | 7.65% | 1.38 |
| +D3R Agonist 1 × 10⁻⁴M | 9,300 | 10.27% | 1.68 |
| +Dopamine | 4,545 | 42.84% | 7.06 |

As to the Sezary T-lymphoma (Table 8). The D1R-agonist caused a dramatic increase in the number of necrotic cells, but also a 2 fold increases in the percent % of apoptotic cells.

TABLE 8
Killing Human Sezary T-Cell Lymphoma (HuT 78)
Cells by D1R, D2R or D3R Agonists or Dopamine

| | No. of | % Necrotic | % Apoptotic |
|---|---|---|---|
| | Cells | Cells | Cells |
| Untreated | 26,250 | 19.02% | 12.28 |
| +D1R Agonist 1 × 10⁻⁴M | 11,835 | 69.75% | 22.97 |
| +D2R Agonist 1 × 10⁻⁴M | 23,880 | 21.97% | 12.00 |
| +D3R Agonist 1 × 10⁻⁴M | 18,975 | 21.40% | 14.67 |
| +Dopamine | 16,395 | 39.80% | 19.07 |

### Example 9

### Effect of D1R Agonists Other than Fenoldopam on TCR-Activated and Resting Normal Peripheral Human T-Cells

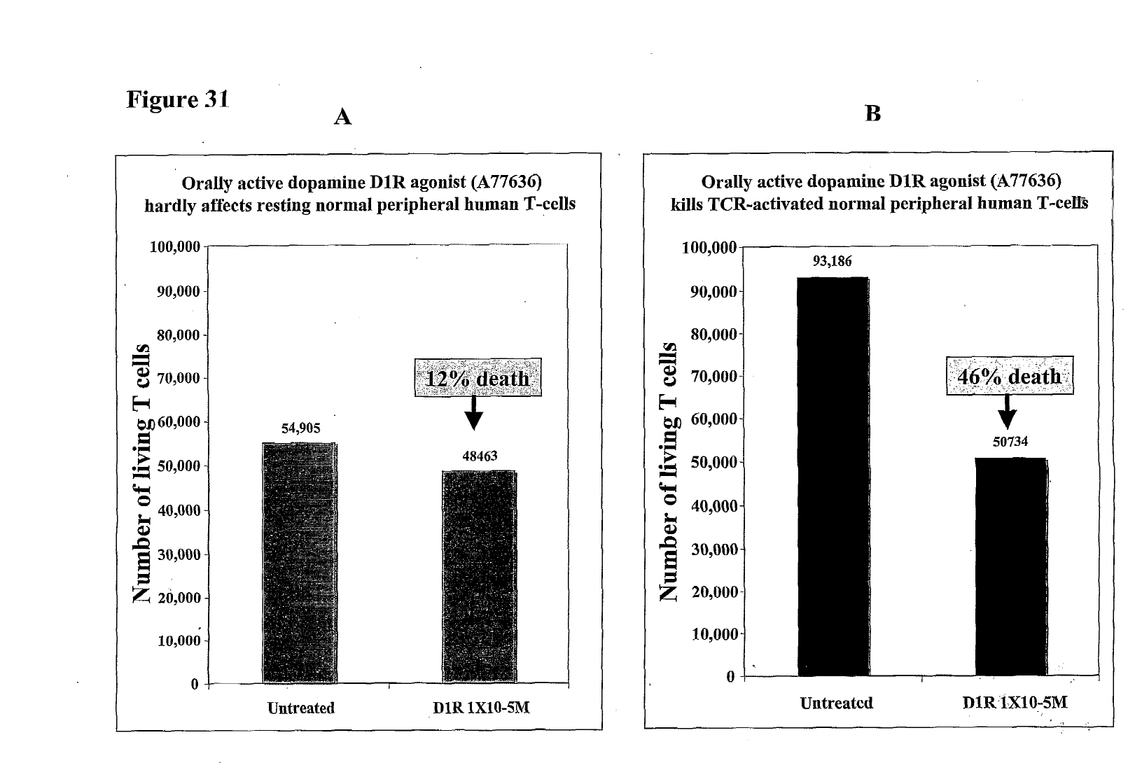

D1R agonists other than fenoldopam also kill much more TCR-activated than resting normal peripheral human T-cells. In line with the elevated levels of D1R expression found herein in TCR-activated normal human T-cells (FIGS. 6 and 7), and the finding that FDM causes marked death of these activated cells (FIG. 8), while hardly affecting the resting normal human T-cells (FIG. 9), other D1R agonists display a similar-property (FIG. 31). Thus, for example, the A77636 highly selective dopamine D1R agonist, used at 10⁻⁵ M, killed 12% of the resting normal human T-cells, and 46% (i.e., 3.8 fold more) of the TCR-activated normal human (FIG. 31).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

1. A method for causing the death of human or other animal cells that express the dopamine D1 receptor, comprising causing said cells to come into contact with an effective amount of a selective dopamine D1 receptor agonist.

2. A method in accordance with claim 1, wherein said cells that express the dopamine D1 receptor are leukemia or lymphoma cells.

3. A method in accordance with claim 1, wherein said cells that express the dopamine D1 receptor are cancer cells that express the dopamine D1 receptor, which cancer cells are other than leukemia or lymphoma cells.

4. A method in accordance with claim 1, wherein said cells that express the dopamine D1 receptor are TCR-activated T-cells.

5. A method in accordance with claim 4, wherein said TCR-activated T-cells are autoimmune T-cells.

6. A method in accordance with claim 1, wherein said step of causing said cells to come into contact with an effective amount of a selective dopamine D1 receptor agonist comprises administering said dopamine D1 receptor agonist into the body of a human or animal subject having a disease or condition that can be alleviated by the elimination of cells that express the dopamine D1 receptor.

7. A method in accordance with claim 6, wherein said disease or condition is a cancer the cells of which express the dopamine D1 receptor.

8. A method in accordance with claim 7, wherein said disease or condition is leukemia or lymphoma and said cells that express the dopamine D1 receptor are leukemia or lymphoma cells.

9. A method in accordance with claim 6, wherein said disease or condition is a T-cell mediated autoimmune disease.

10. A method in accordance with claim 9, wherein said T-cell mediated autoimmune disease is insulin-dependent (type 1) diabetes mellitus, multiple sclerosis, myasthenia gravis, autoimmune myocarditis, alopecia or psoriasis.

11. A method in accordance with claim 6, wherein said disease or condition is one caused or exacerbated by over-activated inflammatory T-cells.

12. A method in accordance with claim 11, wherein said disease or condition is intractable inflammation.

13. A method in accordance with claim 6, wherein said disease or condition is graft versus host disease and said cells that express the dopamine D1 receptor are activated donor versus host T-cells.

14. A method in accordance with claim 6, wherein said disease or condition is graft rejection and said cells that express the dopamine D1 receptor are host T-cells activated against the graft tissue.

15. A method in accordance with claim 6, wherein said administering step is by intravenous, subcutaneous, intraperitoneal, intratumoral, intrathecal, or intracranial injections.

16. A method in accordance with claim 1, wherein said step of causing said cells to come into contact with an effective amount of a selective dopamine D1 receptor agonist comprises contacting said cells with said dopamine D1 receptor agonist ex vivo.

17. A method in accordance with claim 16, wherein said cells are a cell population from which it is desired to purge leukemia, lymphoma or activated T-cells.

18. A method in accordance with claim 17, further including the step of using said purged cell population for bone marrow transplantation, T-cell transplantation, or in vitro culturing to harvest molecules secreted thereby.

19. A method in accordance with claim 16, wherein said cells are autologous T-cells from a human or other animal subject with leukemia or lymphoma.

20. A method in accordance with claim 19, further including the step of administering back to the human or animal subject the autologous T-cells that have been so treated ex vivo, thereby purging said T-cells of leukemia or lymphoma cells.

21. A method in accordance with claim 1, wherein said agonist is a salt of fenoldopam.

22. A method in accordance with claim 21, wherein said agonist is fenoldopam mesylate.

23. A method in accordance with claim 21, wherein said agonist is fenoldopam hydrobromide.

24. A method in accordance with claim 1, wherein said agonist is (1R-cis)-1-(aminomethyl)-3,4-dihydro-3-tricyclo[3.3.1.13,7]dec-1-yl-[1H]-2-benzopyran-5,6-diol hydrochloride.

25. A method in accordance with claim 1, wherein said agonist is (±)-1-phenyl-2,3,4,5-tetrahydro-(1H)-3-benzazepine-7,8-diol hydrobromide.

26. A method in accordance claim 1, wherein said agonist is cis-(±)-1-(aminomethyl)-3,4-dihydro-3-phenyl-1H-2-benzopyran-5,6-diol hydrochloride.
